(19) United States
(12) Patent Application Publication
(10) Pub. No.: US 2009/0325258 A1
(43) Pub. Date: Dec. 31, 2009

(54) Magnetic particle holding carrier and method for preparing the same

(75) Inventors: Tadashi Matsunaga, Tokyo (JP); Haruko Takeyama, Tokyo (JP); Tomoko Yoshino, Tokyo (JP); Hideji Tajima, Chiba (JP)

Correspondence Address: HAYNES AND BOONE, LLP;IP Section, 2323 Victory Avenue, Suite 700, Dallas, TX, 75219, US

(73) Assignees: Tokyo University of Agriculture and Technology, Fuchu-shi (JP); Precision System Science Co,. Ltd, Matsudo-shi (JP)

(21) Appl. No.: 11/921,548

(22) PCT Filed: Jun. 2, 2006
(86) PCT No.: PCT/JP2006/311095

§ 371 Date: Sep. 2, 2009

(30) Foreign Application Priority Data
Jun. 3, 2005 (JP) 2005-164833
Nov. 11, 2005 (JP) 2005-327910

(51) Int. Cl. C12N 11/16; B05D 5/12; B05D 1/04

(52) U.S. Cl. 435/174; 427/127; 427/475

(57) ABSTRACT

Provided is a magnetic particle holding carrier enabling automatization of treatment of a biological substance such as a protein by improving dispersibility of nano-size magnetic particles and suppressing nonspecific adsorption onto the wall of a container such as a pipette tip without damaging the properties of the nano-size magnetic particles such as a large solid-phase area and ability to arbitrarily design a functional protein, and provide a method for preparing the same. The magnetic particle holding carrier is formed of a micro-size nonmagnetic carrier and a plurality of nano-size magnetic particles bound to the carrier.

### TECHNICAL FIELD

The present invention relates to a magnetic particle holding carrier and a method for preparing the same.

### BACKGROUND ART

In conventional techniques for analyzing and treating biological substances such as DNA and proteins, a biological substance is held on magnetic or nonmagnetic particles, which have a large solid-phase surface per volume compared to other solid-phase carriers such as a microtiter plate, mixed in a solution containing a target biological substance and suspended to detect, separate, isolate and extract the target biological substance by use of a high reactivity and encounter probability thereof. Particularly, magnetic particles are an excellent solid-phase carrier having various advantages. For example, they can be easily and quickly collected by external application of a magnetic field, and therefore centrifugation and filtration steps can be removed. Because of such an advantage, magnetic particles can contribute to automatization of a whole reaction process and miniaturization of a reaction apparatus, with the result that a sample-treatment efficiency and test reproducibility can be drastically improved. For this purpose, various types of magnetic particles having different particle sizes and made of different materials are presently available in the market and used in a wide variety of fields, for example, isolation/purification of DNA and mRNA in the field of genomics, isolation/purification of proteins and peptides and analysis of interaction between proteins in the field of proteomics, and drug targeting and detection of pathogenic viruses in the medical field. In addition, an automation system provided with a magnet for collecting magnetic particles has been developed and automatization of treatment steps have been reported in the fields of nucleic acid extraction, peptide extraction and immunoassay.

However, magnetic particles presently available in the market have a functional protein constructed on the surface. However, the type of the functional protein is limited to an antibody, Protein A, Protein G, streptavidin and so forth. To arbitrarily construct a functional protein, for example, silica gel beads are prepared so as to contain a magnetic carrier such as magnetite and the functional protein is electrostatically adsorbed to the inner wall of fine pores thereof. Such a complicated operation must be sometimes performed.

In addition, a specific contrivance was required for maintaining activity of the functional protein.

To develop further a wide range of applications such as analysis for gene function and research for lead compounds of medicinal products in the future, it has been desired to develop a new type of magnetic particles capable of constructing various types of functional proteins without damaging the activity.

On the other hand, bacterial magnetic particles, which are produced by a magnetic bacterial strain, Magnetospirillum magneticum AMB-1, are coated with an organic membrane called a magnetic microparticle membrane containing a phospholipid as a main component. On the magnetic microparticle membrane, various types of membrane proteins are present. As the membrane proteins present on the magnetic microparticle membrane of the AMB-1 strain, MagA protein, MpsA protein and Mms16 protein are identified (e.g., Patent Documents 1, 4 and 5). Furthermore, the present inventors and others have so far reported that various types of proteins can be expressed on the bacterial magnetic particles by fusing a desired protein gene with the 5′ end or 3′ end of a gene encoding a protein present on a lipid bilayer of the bacterial magnetic particles. Examples of these proteins include not only water-soluble proteins such as luciferase (Non-Patent Document 1), acetate kinase, Protein A (Non-Patent Document 2) and estrogen receptor but also transmembrane proteins such as a G protein-conjugated receptor (Non-Patent Document 3). A various applications of these proteins are expected.

The bacterial magnetic particles produced within magnetic bacterial cells or the bacterial cells themselves can be easily separated from a solution by use of a magnet. Therefore, the particles or cells are useful for producing and isolating proteins or collecting, searching, detecting and quantifying various types of substances. By virtue of this feature, automatic immunoassay systems for insulin and endocrine disrupting substances have been constructed using the bacterial magnetic particles having an antibody immobilized thereon. This system has a dispensing apparatus in which a pipette tip made of propylene is provided to a nozzle and a magnet is detachably provided to the tip. By use of the dispensing apparatus, a magnetic particle suspension solution is suctioned or discharged to separate particles from a reagent and continuously a reagent to be used in the next step is suctioned or discharged. In this manner, resuspension can be performed. In this method, since recovery of magnetic particles can be performed on the inner wall of the tip, loss and error taking place when the magnetic particles are separated from a suspension solution can be reduced.

However, in general, it is difficult to magnetically control nano-size magnetic particles in liquid. To explain more specifically, since the content of a magnetic substance in a particle is very small, the magnetic force applied for magnetic separation is very weak. As a result, separation may not be performed quickly.

In addition, since nonspecific adsorption of bacterial magnetic particles to the inner wall of a tip occurs during the separation, resuspension efficiency may further decrease.

This is considered because nano-size magnetic particles, that is, bacterial magnetic particles, have a large surface area/volume ratio. As a result, they are less affected by suction/discharge of a reagent and strongly adsorbed by the tip. In attempts to overcome the problem, various approaches have been made including an approach of increasing hydrophobicity of the wall surface of a pipette tip and an approach of adding a surfactant to a solvent. However, it was very difficult to define conditions for obtaining a stable magnetic separation rate.

Since conditions such as the size of bacterial magnetic particles, etc., strength of magnetic force and magnetism are fixed, when a preparation substance such as a protein using bacterial magnetic particles is subjected to a treatment including separation, extraction and resuspension using a magnetic field or a filter, satisfactory results are not always obtained with respect to automatization, efficiency, handleability, diversification and quickness depending upon the purpose of treatment.

In particular, in the case where separation and extraction are performed by applying a magnetic field to aggregate bacterial magnetic particles and thereafter the magnetic field is removed as mentioned above, it is difficult to release the aggregation of particles and resuspend the particles in a solution by simple removal of the magnetic field. In this case, it is difficult to attain automatization and satisfy efficiency, etc.

Then, a first object of the present invention is to provide a magnetic particle holding carrier enabling automatization of treatment of a biological substance such as a protein by improving dispersibility of nano-size magnetic particles and suppressing nonspecific adsorption onto the wall of a container such as a pipette tip without damaging the properties of the nano-size magnetic particles such as a large solid-phase area and ability to arbitrarily design a functional protein, and provide a method for preparing the same.

A second object of the present invention is to provide a magnetic particle holding carrier enabling a treatment to be carried out easily, efficiently and accurately, while taking advantage of the features of a micro-size carrier, by imparting magnetism to the micro-size nonmagnetic carrier or by increasing the surface area of a solid phase, and provide a method for preparing the same.

A third object of the present invention is to provide a magnetic particle holding carrier applicable to further diversified, generalized, or complicated treatments or a wide variety of treatments by fitting nano-size magnetic particles to various types of carriers and provide a method for preparing the same.

A fourth object of the present invention is to provide a magnetic particle holding carrier capable of stably treating a target biological substance such as protein with a high recovery rate and provide a method for preparing the same.

[Patent Document 1] Japanese Patent Laid-Open No. 8-228782

[Patent Document 2] Japanese Patent Laid-Open No. 10-108689

[Patent Document 3] Japanese Patent Laid-Open No. 11-285387

[Patent Document 4] WO97/35964

[Patent Document 5] Japanese Patent Laid-Open No. 2002-176989

[Patent Document 6] Japanese Patent Laid-Open No. 2004-261169

[Patent Document 7] Japanese Patent Laid-Open No. 2004-290039

[Non-Patent Document 1] Nakamura, T., et al., J. Biochem, 118, 23-7 (1995)

[Non-Patent Document 2] Tanaka, T., et al., Anal. Chem., 72, 3518-22 (2000)

[Non-Patent Document 3] Yoshino, T., et al., Appl. Environ. Microbiol., 70, 2880-5 (2004)

### DISCLOSURE OF THE INVENTION

The present inventors made it possible to automatize a treatment with respect to nano-size magnetic particles much easier by fitting nano-size magnetic particles (about 1 nm to several 100 nm) such as bacterial magnetic particles produced by a magnetic bacterium, to a particulate carrier having a size on the order of micrometer (ranging from about 1 μm to several 100 μm) in accordance with the purpose of the treatment.

More specifically, a first invention is directed to a magnetic particle holding carrier having a micro-size nonmagnetic particulate carrier and a plurality of nano-size magnetic particles held on the carrier. The “particulate carrier” used herein is a solid matter having properties, size and mass which can render the solid to suspend in liquid. The carrier has a size on the order of micrometer, for example, about 1 μm to several 100 μm. The size, mass and material thereof can be determined in accordance with the purpose of a treatment.

In the meantime, examples of the material for the carrier include metals; metal compounds such as a semiconductor, semimetal, and metal oxide; inorganic substances such as ceramic, glass and silica; polymers such as resins including rubber, latex, polystyrene, polypropylene, polyester, and acryl and fiber substances including cellulose and nylon; and organic substances such as naturally occurring substances such as natural fibers including silk. To describe more specifically by taking a fiber substance as an example, mention may be made of all aromatic polyamides made of a “polyamide based polymer”, such as silk, etc., nylon (3-nylon, 6-nylon, 6,6-nylon, 6,10-nylon, 7-nylon, 12-nylon, etc.) and PPTA (polyparaphenyleneterephthalamide) and hetero-ring containing aromatic polymers.

Furthermore, as the carrier, fibrous, porous and gelatinous substances may be mentioned.

The term “holding” used herein means that the magnetic particles are associated with the carrier by binding them directly or indirectly via a different type of substance. Examples of the state of “holding” include binding between a receptor or ligand of a magnetic particle and the corresponding ligand or receptor of a carrier, such as binding between streptavidin and biotin or anti-His antibody and His, and direct binding between a functional group of a carrier and a functional group of a magnetic particle via a covalent bond, hydrogen bond or electrostatic bond.

The term “magnetic particle” used herein is a particle having magnetism and a nano-meter size, for example, from about 1 nm to several 100 nm. The size, mass, material, structure (e.g., a single domain, surface coating with various coating materials) and properties thereof (e.g., paramagnetism, super-paramagnetism, ferromagnetism, ferrimagnetism, magnitude of magnetic force) may be determined depending upon the purpose of a treatment. Examples of the material include iron hydroxide, iron oxide hydrate, iron oxide, iron oxide mixture and iron such as γ-Fe₂O₃ and Fe₃O₄. The magnetic particles may be obtained as bacterial magnetic particles (BMPs) produced intracellularly by, for example, a magnetic bacterium, or obtained by coating the aforementioned material with various coating substances. Examples of the coating substances include organic substances capable of generating various functional groups, ionic substances capable of generating ions, surface-stabilizing substances (such as aliphatic di- and poly-carboxylic acids, substitution products and derivatives thereof) capable of preventing aggregation and precipitation by a magnetic field, specific finding substances (such as ligands and receptors) and pharmacologically active agents.

The bacterial magnetic particle (BMP) is a particle having magnetism and produced intracellularly by a magnetic bacterium. Examples of the magnetic bacterium used herein include microorganisms of the species Magnetospirillum such as Magnetospirillum magneticum AMB-1 (FIRM BP-5458), MS-1 (IFO 15272, ATCC31632, DSM3856) and SR-1 (IFO 15272 DSM6361); and the species Desulfovibrio such as Desulfovibrio sp. RS-1 (FERM P-13283).

By virtue of the structure of a magnetic particle holding carrier having a micro-size nonmagnetic particulate carrier and a plurality of nano-size magnetic particles held on the carrier, magnetism can be imparted to the nonmagnetic carrier. Furthermore, since a plurality of nano-size magnetic particles are massively deposited onto a micro-size nonmagnetic particulate carrier, magnetic force applied to a single particle can be increased. At the same time, since the carrier is a micro-bead and the surface of the micro-bead is covered with nano beads, the surface area of such a structure comes to be increased, with the result that a surface area/volume ratio per particle can be reduced.

A second invention is directed to a magnetic particle holding carrier according to the first invention in which the magnetic particles express or are capable of expressing a predetermined functional peptide or protein.

For example, in the case where the magnetic particles are bacterial magnetic particles, the membrane of each of the bacterial magnetic particles, which serves as a coating substance, more specifically, a lipid bilayer coating the outer surface of a bacterial magnetic particle, has various proteins that have been identified. The protein, which is expressed on the bacterial magnetic particle membrane while partly or wholly bounded thereon, can be used as an anchor protein. The anchor protein plays a role of anchoring a protein fused therewith onto the membrane.

The functional peptide or protein is expressed, for example, by ligating a structural gene encoding the functional peptide or protein downstream of a promoter and introducing such a construct into a bacterial cell. In this manner, a desired peptide or protein can be expressed in a bacterial magnetic particle of a magnetic bacterium.

To describe more specifically, to express a ZZ domain, which is a IgG binding domain of Protein A, in the strain AMB-1, for example, magnetic bacterium, Mms 16 promoter of magnetic bacterium Magnetospirillum magneticum AMB-1 is used as a promoter and Mm 13 is used as an anchor protein, and further, pEZZ18 is used as a gene encoding the ZZ domain.

A third invention is directed to a magnetic particle holding carrier according to the first or second invention, in which the carrier has a ligand or a receptor on the surface thereof and the magnetic particles has the corresponding receptor or ligand and the magnetic particles are held on the carrier by ligand-receptor binding.

The term “ligand” used herein refers to a molecule to which a predetermined receptor is bound. Examples thereof include genetic materials such as a nucleic acid and biological substances such as a protein, sugar, sugar chain and peptide. Specific examples thereof include an agonist and antagonist against a cell membrane receptor of a magnetic bacterial cell, toxic substance (toxin and venom), viral epitope, hormone, hormone receptor, peptide, enzyme, enzyme substrate, lectin, sugar, oligonucleotide, polynucleotide, oligosaccharide and antibody. These may be naturally occurring substances or artificially synthesized substances. The term “receptor” used herein refers to a substance having bindability to the ligand. Examples thereof include genetic materials such as a nucleic acid and biological substances such as a protein, sugar, sugar chain and peptide. More specifically, examples of a ligand-receptor couple include various antigen-antibody combinations such as biotin and avidin; biotin and streptavidin; and Protein A and an antibody. Examples of the antibody include a rabbit-derived anti-goat IgG antibody and a goat-derived anti-mouse IgG antibody. Such a ligand or a receptor is introduced into a carrier or a magnetic particle, for example, by covalently binding the ligand or receptor to a functional group present on the carrier, magnetic particle, a surface thereof or a coating substance such as a film. Alternatively, the receptor or ligand is allowed to express on the carrier, magnetic particle, a surface thereof or a coating substance such as a film.

A fourth invention is directed to a magnetic particle holding carrier according to any one of the first to third inventions, in which the magnetic particles are held on the carrier via a covalent bond, hydrogen bond or electrostatic bond.

The carrier and the magnetic particles are covalently bonded, for example, by hydrolyzing a peptide bond of a material for the carrier such as nylon, or a coating substance applied on the carrier or the magnetic particles, thereby generating a functional group required for immobilizing a biological substance on the surface of the carrier or magnetic particles. Examples of the functional group bindable to a biological substance herein include, a carboxyl group (—COOH), an amino group (—NH₂), a thiol group and a group derived from these. Binding may be made between the same types of functional groups or between different types of functional groups.

When covalent bonding is performed, a chemical bonding method is preferably employed using a crosslinking agent such as EDC (ethylene dichloride) or Sulfo-LC-SPDP (sulfosuccinimidyl-6-(3-[2-pyridyldithio]-propionamido)hexanoate) and Sulfo-SMCC (sulfosuccinimidyl-4-(N-maleimidomethyl)cyclohexane-1-carboxylate). On the other hand, when hydrogen bonding is performed, the carrier must be formed of a substance having atoms X and Y (such as nitrogen, oxygen, phosphorus, sulfur and halogen), which are electrically more negative than a hydrogen atom, or coating the surface of the carrier and the magnetic particles with such a substance, thereby performing binding via a hydrogen atom. Furthermore, when electrostatic bonding is performed, the carrier must be formed of a substance (ionic crystal substance) having a cation and an anion or each of the carrier and magnetic particles must be coated with such a substance.

A fifth invention is directed to a magnetic particle holding carrier according to any one of the first to fourth inventions, in which the magnetic particles have a single type or a plurality of types of marker substances.

The term “marker substance” used herein refers to a substance that magnetic particles held on the carrier have, for example, a substance capable of recognizing a type, property and structure of a protein. Recognition is, for example, optically performed. Examples of an optically detectable maker substance include, fluorescent substances such as Cy3, Cy5, FITC, rhodamine and IRD40; and chemiluminescent substance for use in evaluation of the activity of an antibody-introduced onto the magnetic particles, such as ALP (alkaline phosphatase).

A sixth invention is directed to a magnetic particle holding carrier according to the fifth invention, in which the marker substance has a ligand or a receptor and the magnetic particles have the corresponding receptor or ligand and the marker substance is introduced into the magnetic particles by ligand-receptor binding. The magnetic particles have the corresponding receptor or ligand, for example, on the surface thereof or the coating substance thereof.

A seventh invention is directed to a magnetic particle holding carrier according to the fifth invention or the sixth invention in which the marker substance is introduced into the magnetic particles via a covalent bond, hydrogen bond or electrostatic bond.

An eighth invention is directed to a magnetic particle holding carrier according to the fifth invention or the sixth invention, in which the receptor or ligand is expressed on the magnetic particles.

A ninth invention is directed to a magnetic particle holding carrier according to any one of the first to eighth inventions, in which the magnetic particles are isolated from a magnetic bacterium. Needless to say, the magnetic particles used herein are bacterial magnetic particles.

As another aspect, a tenth invention is directed to a method for preparing a magnetic particle holding carrier having a plurality of nano-size magnetic particles on a macro-size nonmagnetic particulate carrier, comprising a process step for applying processing to the magnetic particles and/or the carrier; and a suspension step for suspending the magnetic particles and a plurality of carriers in liquid.

An eleventh invention is directed to a method for preparing a magnetic particle holding carrier according to the tenth invention, in which the process step has an expression step for expressing a predetermined functional peptide or protein on the magnetic particles.

For example, in the case of the magnetic particles are bacterial magnetic particles, the expression step has a culturing step for culturing a transformant formed by introducing a ZZ domain expression plasmid into the bacterial magnetic particles.

A twelfth invention is directed to a method for preparing a magnetic particle holding carrier according to the tenth invention or the eleventh invention, in which the process step has an introduction-into-carrier step for introducing a ligand or a receptor into the carrier and/or an introduction-into-magnetic-particle step for introducing the corresponding receptor or ligand into the magnetic particles.

For example, in the case of the magnetic particles are bacterial magnetic particles, in the process step, the receptor or ligand to be introduced in the magnetic particles is, for example, the ZZ domain mentioned above, and the corresponding ligand or receptor thereto to be introduced into the carrier is biotin.

A thirteenth invention is directed to a method for preparing a magnetic particle holding carrier according to any one of the tenth to twelfth inventions, in which covalent bonding, hydrogen bonding or electrostatic coupling is performed in the suspension step. For example, to perform covalent bonding, a crosslinking agent is preferably added.

A fourteenth invention is directed to a method for preparing a magnetic particle holding carrier according to any one of the tenth to thirteenth inventions, in which the process step has a step of introducing a marker substance into the magnetic particles.

A fifteenth invention is directed to a method for preparing a magnetic particle holding carrier according to the fourteenth invention, having a step of introducing a ligand or a receptor into the marker substance and/or a step of introducing the corresponding receptor or ligand into the magnetic particles.

A sixteenth invention is directed to a method for preparing a magnetic particle holding carrier according to the fifteenth invention, in which covalent bonding, hydrogen bonding or electrostatic coupling is performed in the process step.

A seventeenth invention is directed to a method for preparing a magnetic particle holding carrier according to the fifteenth invention or the sixteenth invention, in which the process step has a step of expressing the receptor or ligand on the magnetic particles.

An eighteenth invention is directed to a method for preparing a magnetic particle holding carrier according to any one of the tenth to seventeenth inventions, further having an isolation step for isolating the magnetic particles from a magnetic bacterium. Needless to say, the magnetic particles used herein are bacterial magnetic particles.

According to the first invention or the tenth invention, magnetism can be imparted to a nonmagnetic carrier by fitting nano-size magnetic particles to a micro-size nonmagnetic particulate carrier. By virtue of this, the carrier can act as magnetized particles and thus separation, transfer and resuspension of the carrier can be accurately, quickly and easily and further automatically performed without damaging the properties of the micro-size nonmagnetic carrier or the properties of the nano-size magnetic particles and while preventing the magnetic particles from mutually aggregating due to magnetic force.

In addition, the surface area of the micro-size carrier can be increased and thereby a treatment can be further efficiently performed.

When the type of carrier is selected in accordance with a purpose for a treatment, the treatment can be performed in a suitable manner and the treatment can be performed automatically, variously, efficiently, accurately, quickly, and easily.

According to the second invention or the eleventh invention, a predetermined functional peptide or protein is allowed to express on the magnetic particles. Therefore, even if a functional peptide or protein cannot be directly fit to a carrier, the peptide or protein can be fit to the carrier via the magnetic particles. Hence, various substances can be treated automatically, efficiently, accurately, quickly and easily.

According to the third invention or the twelfth invention, the carrier and the magnetic particles can be tightly bound by way of ligand-receptor specific binding.

According to the fourth invention or the thirteenth invention, the carrier and the magnetic particles can be tightly bound by way of, for example, covalent bonding. Particularly, in the case of covalent bonding, the carrier and the magnetic particles can be tightly and easily bound by use of functional groups that the carrier and the magnetic particles have.

According to the fifth invention or the fourteenth invention, labeling can be easily performed in units of carriers by fitting a marker substance to magnetic particles. By virtue of this, various applications, analyses and detections can be performed.

According to the sixth invention or the fifteenth invention, since a marker substance is fitted to a carrier by use of the binding between a ligand and a receptor, labeling of a marker can be tightly performed without fail.

According to the seventh invention or the sixteenth invention, since a marker substance is covalently bound to the magnetic particles, labeling of the marker substance can be performed to each of the carriers in a simple manner.

According to the eighth invention or the seventeenth invention, a protein serving as a receptor is expressed on the magnetic particles and used for binding between the carrier and the magnetic particles or the marker substance and the magnetic particles. Therefore, the receptor is integrated into the magnetic particles into one body, forming tight bonding between the carrier and the magnetic particles or between the magnetic particles and the marker substance.

According to the ninth invention or the eighteenth invention, magnetic particles having a peptide or protein simply and inexpensively expressed thereon can be prepared by isolating the bacterial magnetic particles from a magnetic bacterium having a gene encoding a functional peptide or protein introduced thereto. As a result, various types of magnetic particle holding carriers can be prepared.

### BEST MODE FOR CARRYING OUT THE INVENTION

FIG. 1 schematically shows magnetic particle holding carriers 11, 21, 31 and 41 according to the first to fourth embodiments of the present invention.

As shown in FIG. 1(a), the magnetic particle holding carrier 11 according to the first embodiment has a micro-size nonmagnetic particulate carrier 12 (for example, Streptavidin Coated Beads, 1 μm YG manufactured by Polysciences, Ink.), which has a particle size of about 1 μm and is formed of latex whose surface is coated with streptavidin 13 serving as the receptor; a nano-size super-magnetic single domain particle(s) 14, which serves as the magnetic particles and is coated with a substance having biotin 15 serving as a ligand and an amino group; and a fluorescent dye 16 (Cy3-NHS) introduced to the amino group. Note that the super-magnetic single domain particle is disclosed, for example, in WO96/03653 or WO97/35200.

As shown in FIG. 1(b), a magnetic particle holding carrier 21 according to a second embodiment has a micro-size nonmagnetic particulate carrier 22 (for example, Streptavidin Coated Beads, 1.00 μm manufactured by Polysciences, Ink.) which has a particle size of about 1 μm and is formed of latex whose surface is coated with a substance having an amino group 23 serving as a functional group; a nano-size super-magnetic single domain particle(s) 24, which serves as the magnetic particles and coated with a substance having a carboxyl group 27 serving as a functional group; and a fluorescent dye 26 (for example, Cy3) introduced into the carboxyl group 27. The amino group 23 of the particulate carrier 22 and the carboxyl group 27 of the super-magnetic single domain particle 24 are connected by a chemical bonding method using a crosslinking agent EDC 28 (ethylene dichloride).

FIG. 1(c) shows a magnetic particle holding carrier 31 according to a third embodiment. Like reference numerals are used to designate like structural elements corresponding to those like in FIG. 1(a). The magnetic particle holding carrier 31 has a micro-size nonmagnetic particulate carrier 32 (for example, Streptavidin Polystyrene Particles manufactured by Spherotech, Inc.) which has a particle size of 5.0 to 5.9 μm and is formed of polystyrene and whose surface is coated with streptavidin 13 serving as a receptor; a bacterial magnetic particle(s) (BMPs) 33, which serves as the magnetic particles; a fluorescent dye 36, which is Cy3 bisNHS ester (purchased from Amercham Biosciencs) introduced to the bacterial magnetic particle(s) 33; and biotin 34 formed of Sulfo-NHS-LC-LC-biotin and introduced in the bacterial magnetic particle(s) 33.

The bacterial magnetic particle(s) 33 having biotin 34 and a fluorescent dye 36 introduced thereto will be referred to as a biotin/fluorescent dye-introduced bacterial magnetic particle(s) 37.

FIG. 1(d) shows a magnetic particle holding carrier 41 according to a fourth embodiment. Like reference numerals are used to designate like structural elements corresponding to those like in FIG. 1(c) and any further explanation is omitted. The magnetic particle holding carrier 41 has a micro-size particulate carrier 32, which is formed of polystyrene and whose surface is coated with streptavidin 13; a bacterial magnetic particle(s) 33, which serves as the magnetic particles; a fluorescent dye 36 consisting of Cy3 bisNHS ester introduced into the bacterial magnetic particle(s) 33; biotin 34 consisting of Sulfo-NHS-LC-LC-biotin; and an antibody 35 consisting of a rabbit-derived anti-goat IgG antibody and introduced into the bacterial magnetic particle(s) 33. The antibody 35 used herein is a functional protein. Introduction of the antibody 35 into the bacterial magnetic particle 33 enables the magnetic particle holding carrier to hold a predetermined substance having an antigen specifically reacting with the antibody 35. The relationship between the antibody 35 and the antigen is the same as that between a receptor and a ligand. Furthermore, the bacterial magnetic particle 33 having biotin 34 and the fluorescent dye 36 introduced thereto and further having the antibody 35 immobilized therein will be referred to as an antibody-immobilized biotin/fluorescent dye-introduced bacterial magnetic particle(s) 39.

FIG. 1(e) shows a magnetic particle holding carrier 81 according to a fifth embodiment. Like reference numerals are used to designate like structural elements corresponding to those like in FIGS. 1(b), 1(c) or 1(d) and any further explanation is omitted. The magnetic particle holding carrier 81 has a micro-size nonmagnetic particulate carrier 82 (amino group displayed polystyrene micro bead) (AP-60-10 of a diameter of 6 to 8 μm, manufactured by Spherotech Inc.), which is formed of polystyrene whose surface is coated with an amino group 83 serving as a functional group; a bacterial magnetic particle(s) 84 which serves as the magnetic particle(s) and having an amino group 87 serving as a functional group on the surface; and a fluorescent dye 36, (Cy3 bisNHS ester) introduced into the bacterial magnetic particle(s) 84. The particulate carrier 82 and the bacterial magnetic particle(s) 84 are bound by a chemical bonding method using a crosslinking agent 88 (Sulfo-LC-SPDP and Sulfo-SMCC).

Subsequently, a method for preparing a magnetic particle holding carrier 31 according to the third embodiment will be described.

As shown in FIG. 2, the magnetic particle holding carrier 31 is prepared by preparing bacterial magnetic particles 33 serving as the magnetic particles in a step S1, introducing biotin 34 and a fluorescent dye 36 into the bacterial magnetic particles 33 in a step S2, and binding biotin/fluorescent dye-introduced bacterial magnetic particles 37 having biotin 34 and a fluorescent dye 36 introduced thereto to a micro-size particulate carrier formed of polystyrene and labeled with streptavidin 13.

In the step S1, Magnetospirillum magneticum AMB-1 was inoculated to 4.5 L of MSGM (magnetic spirillum growth medium; Blakemore et al. J. Bacteriol 1979, 140:720-729). Aeration with argon gas was performed for 15 minutes to render the conditions slightly aerobic and then stationary culture was performed at room temperature for about 5 days. The bacterial cells cultured were centrifugally collected at 8000 rpm and 4° C. for 8 minutes, suspended in 45 ml of phosphate buffered saline (PBS, pH 7.4), and crushed by a French press at 2000 kg/cm². The crushed cell solution was placed in a conical flask and a neodymium-boron (Nd—B) magnet was attached to the bottom of the flask to magnetically separate the bacterial magnetic particles 33 and then washed in 2-[4-Hydroxyethyl]-1-piperazinyl]ethanesulfonic acid (HEPES) buffer solution (10 mM, pH 7.4) 10 times by an ultrasonic cleaner. The bacterial magnetic particles 33 thus washed were suspended in PBS and stored at 4° C.

On the other hand, to perform immunological assay, a transformant was prepared by introducing a ZZ domain expression plasmid pUM13ZZ into a magnetic bacterium, Magnetospirillum magneticum AMB-1, and cultured to prepare bacterial magnetic particles 33 (ZZ-BMPs) displaying the ZZ domain, which serves as an immunoglobulin G(IgG) binding site of a protein AB-domain, on a lipid bilayer.

The ZZ domain expression plasmid pUM13ZZ is a vector capable of expressing the ZZ domain, which is a Protein A IgG binding domain in the AMB-1 strain. Plasmid pMC18 (Ampr) described in Patent Document 3 formed of pMS-T1 and pUC18 was digested with SspI. An Mms13 gene of a magnetic bacterium, Magnetospirillum magneticum AMB-1 serving as a promoter was obtained by PCR from AMB-1 genome based on a known sequence. Furthermore, a gene encoding a ZZ domain was obtained from pEZZ18 (manufactured by Amersham Biosciences). These PCR products were introduced in the aforementioned plasmid and further, an Mms16 promoter sequence was introduced and then an Mms13 coding sequence and a ZZ domain coding sequence were inserted downstream of the Mms16 promoter sequence so as to fall within a frame. In this manner, Mms13-ZZ domain expression plasmid pUM13ZZ was constructed.

In plasmid pUM13ZZ, an EZZ gene encoding the ZZ domain is fused at the C terminal of the Mms13 gene expressed on the lipid bilayer of the bacterial magnetic particles 33. The Mms13 and EZZ fusion gene is controlled by the promoter of an Mms16 gene. Since a recombinant has an ampicillin resistance, stationary culture was performed in MSGM medium containing 5.0 μg/ml ampicillin for about 7 days. When the bacterial cells were crushed by a French press, a protease inhibitor was added in order to protect the ZZ domain displayed on the bacterial magnetic particles 33.

Next, in the step S2, biotin 34 and fluorescent dye 36 are introduced into bacterial magnetic particles 33.

To a carbonate buffer solution, biotin 34 (Sulfo-NHS-LC-LC-biotin), the fluorescent dye 36 (Cy3 bis NHS ester) were dissolved so as to obtain a concentration of 0.35 mM, and 0.035 mM, respectively to obtain a solution mixture. To 1 ml of the solution mixture, 1 mg of the bacterial magnetic particles 33 was suspended and a reaction was performed at room temperature for one hour while maintaining a dispersion state by ultrasonically stirring the solution at intervals of 5 minutes. Thereafter, the resultant particles were washed 3 times with 1 ml of PBS to obtain biotin/fluorescent dye-introduced bacterial magnetic particles 37 (Cy3-BMP-biotin). The biotin/fluorescent dye-introduced bacterial magnetic particles 37(Cy3-BMP-biotin) were suspended again in 1 ml of PBS and stored at 4° C.

Further in the step S3, the biotin/fluorescent dye-introduced bacterial magnetic particles 37 having the fluorescent dye 36 and biotin 34 introduced thereto are allowed to bind onto the micro-size particulate carrier 32. To bind them, to a suspension solution (3.0×10⁶ beads/ml, 500 μl) of the particulate carrier 32 labeled with streptavidin, the biotin/fluorescent dye-introduced bacterial magnetic particles 37 (50 μg/ml, 100 μl) having the fluorescent dye 36 and biotin 34 introduced thereto were added and pipetting was repeated 10 times for maintaining the dispersion state for 15 minutes. In this manner, the biotin/fluorescent dye-introduced bacterial magnetic particles 37 were constructed on the particulate carrier 32 to prepare the magnetic particle holding carrier 31.

Microscopic observation and flow cytometric analysis of the magnetic particle holding carrier 31 thus prepared are shown in FIG. 3.

In the process for preparing the magnetic particle holding carrier 31, a suspension solution of the biotin/fluorescent dye-introduced bacterial magnetic particles 37 (Cy3-BMP-biotin) was added successively in a plurality of times: 0, 3, 5, 7, 8, 9, 10 (the number of binding steps). After each addition time, the particles were observed by a fluorescent microscope. Furthermore, the distribution of fluorescent intensity was analyzed by flow cytometry to obtain a histogram in which the vertical axis represents the number of cases and the horizontal axis represents relative fluorescent intensity. Moreover, the magnetic particle holding carrier 31 prepared was washed 3 times with distilled water and thereafter observed by a scanning electron microscope (SEM).

Fluorescent microscopic images of the micro-size particulate carriers 32 at the times when the biotin/fluorescent dye-introduced bacterial magnetic particles 37 (Cy3-BMP-biotin) were added 0, 3, 5, 7, 8, 9, times to the streptavidin-labeled micro-size particulate carrier 32, are shown in FIG. 3(1)(a), and the results analyzed by a flow cytometer (FACS) are shown in FIG. 3(1)(b). Based on the results of the flow cytometric analysis, a change of the intensity distribution of fluorescence emitted from the micro-size particulate carrier 32 was observed for each addition time. As a result, it was found that as the number of addition times of the suspension solution of the biotin/fluorescent dye-introduced bacterial magnetic particles 37 (Cy3-BMP-biotin) increases, the peak of the histogram shifts toward the right hand side and the shape of the peak becomes sharp. In addition, the observed change of the peak after the 8th addition time was less. It is considered that the shift of the fluorescence intensity indicates that the amount of the biotin/fluorescent dye-introduced bacterial magnetic particles 37 (Cy3-BMP-biotin) constructed on the micro-size particulate carrier 32 increases, and that the sharp peak indicates quantitative variation of the biotin/fluorescent dye-introduced bacterial magnetic particles 37 between the micro-size particulate carriers 32. From the results, it was demonstrated that the biotin/fluorescent dye-introduced bacterial magnetic particles 37 (Cy3-BMP-biotin) can be efficiently constructed on the micro-size particulate carrier 32 by adding the biotin/fluorescent dye-introduced bacterial magnetic particles 37 (Cy3-BMP-biotin) successively 10 times.

Furthermore, the observation of the magnetic particle holding carrier 31 by a scanning electron microscope (SEM) indicated that the biotin/fluorescent dye-introduced bacterial magnetic particles 37 (Cy3-BMP-biotin) are constructed on the surface of the magnetic particle holding carrier 31, compared to the particulate carrier 32 formed of polystyrene beads to which the biotin/fluorescent dye-introduced bacterial magnetic particles 37 (Cy3-BMP-biotin) are not successively added. FIG. 3(2)(a)(b) each show an illustration based on a further magnified observation image of a single particulate carrier 32, that is, a microphotograph of 1500× magnification. FIG. 3(2)(c) shows an illustration based on based on a further more magnified observation image of the single particulate carrier 32, that is, a microphotograph of 100000× magnification. As is apparent from observation images of these illustrations, biotin/fluorescent dye-introduced bacterial magnetic particles 37 (Cy3-BMP-biotin) do not form a large aggregation on a part of the surface of the micro-size particulate carrier 32 but form a continuous chain over the entire surface of the particulate carrier 32. From this, a high activity is expected when bacterial magnetic particles (BMPs) having a functional protein displayed thereon are constructed.

Next, the magnetic particle holding carrier 31 thus prepared is evaluated for a magnetic separation ratio when a magnetic field is manually applied to the container.

As shown in FIG. 4, a suspension solution of the magnetic particle holding carrier 31 prepared was centrifuged to obtain a 30 μl solution of 5.0×10⁷ beads/ml. In a step S11, a suspension solution 51 having the magnetic particle holding carrier 31 suspended in liquid was placed in a PCR tube 50. In a step S12, an Nd—B magnet 52 was brought into contact with the upper wall surface of the tube 50 to perform magnetic separation for 5 minutes. In a step S13, the supernatant was removed. In a step S14, 30 μl of a fresh PBS was placed. In a step S15, the concentration of beads was determined by use of a hemocytometer before and after magnetic separation, and then, a magnetic separation rate below was calculated:

Bead concentration [B] after magnetic separation/Bead concentration [A] before magnetic separation×100%.

The same operation was repeated using a particulate carrier 32 having no biotin/fluorescent dye-introduced bacterial magnetic particles 37 (Cy3-BMP-biotin) added thereto. The magnetic separation ratios were compared.

FIG. 5 shows measurement results of the magnetic separation ratio. The magnetic separation ratio of the magnetic particle holding carrier 31 prepared was 93.9%, which was same as the result of magnetic beads, Dynabeads (trade name: Dynal, Biotech, streptavidin-labeled superparamagnetic particles having a particle size of 2.7 μm). From this, it was demonstrated that magnetism can be imparted to the micro-size particulate carrier 32 formed of nonmagnetic polystyrene bead by adding the bacterial magnetic particles (BMPs) 33, which is a ferrimagnet, sequentially, and the carrier can be magnetically separated from the suspension solution.

Next, based on FIG. 20, the stability of the magnetic particle holding carrier 31 according to the third embodiment is evaluated as follows.

To 5 mg of bacterial magnetic particles (BMPs) 33, a carbonate buffer solution (pH 8.5) containing 0.35 mM biotin 34 (Sulfo-NHS-LC-LC-biotin) and 0.035 m fluorescent dye 36 (Cy3 bis NHS ester) was added in an amount of 5 ml. A reaction was performed at room temperature for one hour while maintaining a dispersion state by ultrasonically stirring at intervals of 5 minutes. Thereafter, the resultant solution was washed 4 times with 5 ml of PBS to obtain biotin/fluorescent dye-introduced bacterial magnetic particles 37 (Cy3-BMP-biotin).

A suspension solution (50 μg/ml, 8 ml) of the biotin/fluorescent dye-introduced bacterial magnetic particles 37 was added to a suspension solution (3.0×10⁶ beads/ml, 4 ml) of the particulate carrier 32 (polyethylene beads) labeled with streptavidin. A reaction was performed for 15 minutes while maintaining a dispersion state by pipetting. This operation was repeated 10 times to obtain the magnetic particle holding carrier 31.

The cases where beads of the magnetic particle holding carrier 31 (1×10⁶ particles) thus prepared were suspended in 100 μl of PBS buffer solution (10 mM KH₂PO₄, 1.8 mM Na₂HPO₄, 140 mM NaCl, 2.7 mM KCl, pH 7.4), a HEPES buffer solution (10 mM, pH 7.4) and a Tris hydrochloride buffer solution (100 mM, pH 7.0) are shown in FIG. 20(a). The cases where the beads were suspended in the PBS buffer solutions prepared at pH of 2, 4, 6, 8, and 10 are shown in FIG. 20(c). The cases where the beads were suspended in 10×-, 100×- and 100×-dilution PBS buffer solutions, and in distilled water are shown in FIG. (20 (b)). These suspension solutions each were allowed to stand still at room temperature for one hour and 48 hours. Thereafter, the suspension solutions were centrifugally separated at 9100 G for 10 minutes. The precipitation fraction thus obtained was suspended in a PBS buffer solution and observed by a fluorescent microscope.

As a result, as is shown in fluorescent images of the magnetic particle holding carriers 31 shown in FIG. 20, biotin/fluorescent dye-introduced bacterial magnetic particles 37 (Cy3-BMP-biotin) were observed to accumulate on the particulate carrier 32. From this, it was demonstrated that the magnetic particle holding carrier prepared through a biotin-streptavidin reaction is present as a stable complex in broad ranges of pH and a salt concentration and in various types of buffer solutions.

Next, a method for preparing the magnetic particle holding carrier 41 according to the fourth embodiment will be described.

First, whether an antibody 35 can be introduced into the biotin/fluorescent dye-introduced bacterial magnetic particles 37 which are formed by introducing biotin 34 and a fluorescent dye 36 into the bacterial magnetic particle 33, will be checked.

As shown in a step S20, a bacterial magnetic particle 33′ has a ZZ domain 38 displayed. In a step S21, to the bacterial magnetic particle 33′ (ZZ-BMPs) having a ZZ domain 38 displayed thereon, solution mixtures, which were different in concentration and contain the biotin 34 (Sulfo-NHS-LC-LC-biotin) and a fluorescent dye 36 (Cy3 bis NHS ester) in a constant molecular ratio of 10:1, were added. In the same operation as in the method of introducing biotin and a fluorescent dye (Cy3) into the bacterial magnetic particles 33, biotin and the fluorescent dye were introduced into the bacterial magnetic particles 33′ to prepare biotin/fluorescent dye-introduced bacterial magnetic particles 37′ (Cy3-[ZZ-BMP]-biotin). In a step S22, to 50 μg of the biotin/fluorescent dye-introduced bacterial magnetic particles 37′ (Cy3-[ZZ-BMP]-biotin), 50 μl of a solution containing 10 μg/ml antibody 35 (rabbit-derived anti-goat IgG antibody) was added. A reaction was performed for 30 minutes while stirring the solution by an ultrasonic cleaner at intervals of 5 minutes to immobilize the antibody 35. In this manner, antibody-immobilized biotin/fluorescent dye-introduced bacterial magnetic particles 39 (Cy3, biotin-[ZZ-BMP]-Antibody) were prepared. Thereafter, in a step S23, the particles obtained were washed 3 times with 50 μl of PBS and 50 μl of an 8 μg/ml solution of marker antigen 40 (ALP-labeled goat-derived anti-mouse IgG antigen) was added. In a step S24, to 50 μl of a suspension solution containing the antibody-immobilized biotin/fluorescent dye-introduced bacterial magnetic particles 39 (Cy3, biotin-[ZZ-BMP]-Antibody) having the marker antigen 40 bound thereto, 50 μl of lumiphos 530 was added. In a step S25, light emission after 10 minutes was measured and the amount of the marker antigen 40 (ALP-labeled goat-derived anti-mouse IgG antibody) bound was calculated.

The measurement results of emission intensity are shown in FIG. 7. From this, it was found that as the concentration of the solution mixture of biotin 34 (Sulfo-NHS-LC-LC-biotin) and the fluorescent dye 36 (Cy3 bis NHS ester) increases, the amount of the marker antigen 40 bound thereto decreases. It has been reported that an amino acid such as asparagine, glutamine or lysine having an amino group at a side chain is present at an activation site of B-domain of Protein A (Gouda et. al 1998, FIGS. 3 to 6). These amino acids are present in the ZZ domain having a Protein A-simulated amino acid sequence. Therefore, they are conceivably involved in binding with the IgG Fc site as is in the case of Protein A. The reason why the amount of the marker antigen 40 to be bound decreases as the concentration of the solution mixture of biotin 34 (Sulfo-NHS-LC-LC-biotin) and the fluorescent dye 36 (Cy3 bis NHS ester) increases was considered as follows. When biotin 34 (Sulfo-NHS-LC-LC-biotin) or the fluorescent dye 36 (Cy3 bis NHS ester) is bound to the side-chain amino acid of these amino acids, the binding between the ZZ domain and an antibody is sterically hindered, with the result that the amount of the antibody 35 (rabbit-derived anti-goat IgG antibody) to be bound to the bacterial magnetic particles 33 (ZZ-BMPs) decreases. Consequently, the amount of the marker antigen 40 to be bound decreases. The concentrations of the solution mixtures of biotin 34 (Sulfo-NHS-LC-LC-biotin) or the fluorescent dye 36 (Cy3 bis NHS ester) to be employed in this embodiment are 0.35 mM and 0.035 mM. In these cases, it was found that 95% antigen binding activity is maintained. From this, it was suggested that biotin and Cy3 can be introduced without damaging the activity of the ZZ domain, and that the magnetic particle holding carrier 41 can be prepared by use of the antibody-immobilized biotin/fluorescent dye-introduced bacterial magnetic particles 39, thereby constructing a full automatic immune measurement system for a biological substance.

As a next step, since it was elucidated that the antibody can be immobilized onto the biotin/fluorescent dye-introduced bacterial magnetic particles 37 (Cy3-[ZZ-BMP]-biotin), the magnetic particle holding carrier 41 is prepared by use of the antibody-immobilized biotin/fluorescent dye-introduced bacterial magnetic particles 39 (Cy3, biotin-[ZZ-BMP]-Antibody).

To 0.5 mg of the biotin/fluorescent dye-introduced bacterial magnetic particles 37 (Cy3-[ZZ-BMP]-biotin), which were prepared by labeling bacterial magnetic particles (ZZ-BMPs) with biotin and Cy3 in the same manner as in the bacterial magnetic particles 33, 0.5 ml of a solution of 10 μg/ml antibody 35 (rabbit-derived anti-goat IgG antibody) was added. A reaction was performed at room temperature for 30 minutes to immobilize the antibody 35. A suspension solution of the antibody-immobilized biotin/fluorescent dye-introduced bacterial magnetic particles 39 (Cy3, biotin-[ZZ-BMP]-Antibody) and a suspension solution of the biotin/fluorescent dye-introduced bacterial magnetic particles 37(Cy3-[ZZ-BMP]-biotin) having no antibody immobilized thereto were successively added to the beads of the micro-size particulate carrier 32 (streptavidin-labeled micro beads) in the same method as mentioned above to prepare a magnetic particle holding carrier 41, which was subjected to a fluorescent microscopic observation and flow cytometric analysis for fluorescent intensity distribution. To describe more specifically, the activity of the antibody 35 of the magnetic particle holding carrier 41 obtained was checked by immobilizing the antibody 35 at various concentrations onto the biotin/fluorescent dye-introduced bacterial magnetic particles 37 (Cy3-[ZZ-BMP]-biotin) and measuring the emission intensity after the marker antigen 40 was introduced. The measurement results are shown in FIG. 7. According to the measurement results, it was found that as the concentration of the solution mixture of biotin 34 (Sulfo-NHS-LC-LC-biotin) and the fluorescent dye 36 (Cy3 bis NHS ester) increases, the amount of the marker antigen 40 bound thereto decreases. It has been reported that an amino acid such as asparagine, glutamine or lysine having an amino group at a side chain is present at an activation site of B-domain of Protein A (Gouda et. al 1998). These amino acids are present in the ZZ domain having a Protein A-simulated amino acid sequence and conceivably involved in binding with the IgG Fc site as is in the case of Protein A. The reason why the amount of the marker antigen 40 bound thereto decreases as the concentration of the solution mixture of biotin 34 (Sulfo-NHS-LC-LC-biotin) and the fluorescent dye 36 (Cy3 bis NHS ester) increases was considered as follows. When biotin 34 (Sulfo-NHS-LC-LC-biotin) or the fluorescent dye 36(Cy3 bis NHS ester) is bound to the side-chain amino acid of these amino acids, the binding between the ZZ domain and antibody is sterically hindered, with the result that the amount of the antibody 35 (rabbit-derived anti-goat IgG antibody) to be bound to the bacterial magnetic particles 33 (ZZ-BMPs) decreases. Consequently, the amount of the marker antigen 40 to be bound decreases.

As a result, when the concentrations of solution mixtures of biotin 34 (Sulfo-NHS-LC-LC-biotin) or the fluorescent dye 36(Cy3 bis NHS ester) are 0.35 mM and 0.035 mM, 95% antigen binding activity is maintained. From this, it was suggested that biotin and Cy3 can be introduced without damaging the activity of the ZZ domain and that the magnetic particle holding carrier 41 can be prepared by use of the antibody-immobilized biotin/fluorescent dye-introduced bacterial magnetic particles 39, thereby constructing a fullautomatic immune assay system for a biological substance.

FIG. 8 shows micrographic observations and flow cytometric evaluation results of the magnetic particle holding carrier 41. In this example, the magnetic particle holding carrier 41 (FIG. 8(a)) and the magnetic particle holding carrier 31 (FIG. 8(b)), which were respectively prepared by a suspension solution of antibody-immobilized biotin/fluorescent dye-introduced bacterial magnetic particles 39 (Cy3, biotin-[ZZ-BMP]-Antibody) and a suspension solution of the biotin/fluorescent dye-introduced bacterial magnetic particles 37 (Cy3-[ZZ-BMP]-biotin) having no antibody immobilized, and the particulate carrier 32 (FIG. 8(c)) were observed by a fluorescent microscope. As a result, formation of them was observed in any one of the cases. Furthermore, fluorescent intensity histogram was prepared based on flow cytometry. As a result, peaks having the same relative intensity were obtained. From this, it was demonstrated that the antibody 35 immobilized onto the biotin/fluorescent dye-introduced bacterial magnetic particles 37 (Cy3-[ZZ-BMP]-biotin) do not inhibit preparation of the magnetic particle holding carrier 41 using the streptavidin-labeled micro-size particulate carrier 32.

Subsequently, the activity of the antibody 35 immobilized onto the magnetic particle holding carrier 41 to which the antibody thus prepared immobilized thereon is checked.

As shown in FIG. 9, in a step S31, a suspension solution (1.0×10⁸ beads/ml, 20 μl) of the magnetic particle holding carrier 41 having the antibody 35 immobilized thereon is prepared. In a step S32, solutions (20 μl) of the marker antigen 40 (ALP-labeled goat-derived anti-mouse IgG antigen) prepared so as to have final concentrations of 8, 4, 0.8, 0.4, 0.08, 0.04, 0.008, 0.004 μg/ml are added. Each of the reactions is performed at room temperature for 30 minutes. In a step S33, an Nd—B magnet is brought into contact with the tube for 5 minutes to magnetically collect the beads, which are washed 3 times with 40 μl of PBS. In a step S34, 50 μl of lumiphos 530 serving as a luminescent substrate is added. After 20 minutes, in a step S35, emission intensity was measured.

The calculation results of the amount of the marker antigen 40 bound based on the emission intensity are shown in FIG. 10. As is shown in FIG. 10, the amount of the marker antigen 40 bound to the antibody 35 immobilized onto the magnetic particle holding carrier 41 increases depending upon the concentration of the marker antigen 40 added thereto. From the results, it was demonstrated that the antibody 35 immobilized onto the magnetic particle holding carrier 41 has antigen recognition ability and that the magnetic particle holding carrier 41 having the antibody 35 immobilized thereon can be used for immunoassay.

### [Experiment 1]

Immunoassay for detecting a prostate specific antigen (PSA) using a magnetic particle holding carrier according to an embodiment of the present invention is prepared (manually performed).

(1) Study on the Concentration of Antibody Immobilized onto the Bacterial Magnetic Particles (BMPs)

In a step 41 of FIG. 11, the bacterial magnetic particles 33′ (ZZ-BMPs) displaying ZZ domains 38, which are an IgG binding sites of Protein A and obtained from a ZZ domain expressing strain were labeled with the fluorescent dye 36 (Cy3) and biotin 34. In a step S42, to 20 μg of the biotin/fluorescent dye-introduced bacterial magnetic particles 37′ (Cy3-[ZZ-BMP]-biotin), solutions of mouse-derived anti-human PSA antibody 60 (IgG₂ₐ) different in concentration (0 to 60 μg/ml, 20 μl) were added and incubation was performed while stirring at room temperature for one hour to immobilize the antibody 60. The antibody-immobilized biotin/fluorescent dye-introduced bacterial magnetic particles 61 were washed 3 times with PBS. Thereafter in a step S43, alkaline phosphatase (ALP)-labeled goat-derived anti-mouse IgG antibody 62 (10 μg/ml, 20 μl) was added and incubation was performed while stirring at room temperature for 30 minutes. After the incubation product was washed 3 times with PBS, lumiphos 530 (3.3×10⁻⁴ mol/l, 80 μl) was added to measure emission intensity with respect to the antibody-immobilized biotin/fluorescent dye-introduced bacterial magnetic particles 61 prepared by use of solutions of the antibody 60 different in concentration.

As a result, as is shown in FIG. 12, when immobilization was performed by use of an antibody solution, as the concentration of antibody to be added to the biotin/fluorescent dye-introduced bacterial magnetic particles 37′ (Cy3-[ZZ-BMP]-biotin) increased, the emission intensity increased. From this, it was demonstrated that the amount of antibody-immobilized onto the biotin/fluorescent dye-introduced bacterial magnetic particles 37′ (Cy3-[ZZ-BMP]-biotin) can be increased by use of a high concentration antibody solution. The emission intensity reached saturation when an antibody solution (40 μg/ml) was used. Therefore, when the antibody is immobilized onto the biotin/fluorescent dye-introduced bacterial magnetic particles 37′ (Cy3-[ZZ-BMP]-biotin) hereinafter, the concentration of an antibody solution will be set at 40 μg/ml.

(2) Study on the Concentration of the ALP-Labeled Antibody

As shown in FIG. 11, in a step S42, to 1 mg of the biotin/fluorescent dye-introduced bacterial magnetic particles 37′ (Cy3-[ZZ-BMP]-biotin), the mouse-derived anti-human PSA antibody 60 (40 μg/ml, 1 ml) was added. The mixture was stirred at room temperature for one hour to immobilize the antibody to prepare the antibody-immobilized biotin/fluorescent dye-introduced bacterial magnetic particles 61. In a step S44, the suspension solution of the antibody-immobilized biotin/fluorescent dye-introduced bacterial magnetic particles 61 (Cy3-[ZZ-BMP]-biotin) was successively added to streptavidin-labeled polystyrene beads of 5 μm in particle size serving as a particulate carrier 32 to prepare beads of an antibody-immobilized magnetic particle holding carrier 63. In a step S45, to the beads of antibody-immobilized magnetic particle holding carrier 63 (2.0×10⁶ beads), PSA (antigen) 64, (400 μg/ml, 40 μl) was added. The mixture was stirred at room temperature for 30 minutes and incubated to prepare beads of PSA-bound antibody-immobilized magnetic particle holding carrier 65. The beads of the carrier 65 were washed 3 times with PBS. Thereafter, in a step S46, solutions (40 μl) of ALP-labeled mouse-derived anti-human PSA antibody 66 different in concentration were added. The mixture was stirred at room temperature for 30 minutes and incubated to prepare beads of an ALP-labeled PSA bound antibody-immobilized magnetic particle holding carrier 67. After the beads were washed 3 times with PBS, the concentration of beads was determined. The beads (1.0×10⁶) were suspended in 50 μl of PBS. To the suspension solution, lumiphos 530 (3.3×10⁻⁴ mol/l, 50 μl) was added to measure emission intensity. Furthermore, as a control, the same operation was repeated without adding PSA.

Note that, the ALP-labeled mouse-derived anti-human PSA antibody 66 can be obtained, as shown in the box of FIG. 11, by reducing the mouse-derived anti-human PSA antibody (IgG₁) 60 with a reducing agent in a step S47 and reacted with an SH-reactive ALP in a step S48.

As a result, as shown in FIG. 12, the emission intensity increased as the concentration of ALP-labeled antibody to be added in the presence of PSA and reached saturation when 20 μg/ml antibody solution was used. From this, it was considered that the amount of ALP-labeled antibody to be specifically bound to the beads through an antigen-antibody reaction increased by use of a high concentration ALP-labeled antibody solution and reached saturation when 20 μg/ml antibody solution was used.

On the other hand, the same operation was performed in the absence of PSA. As a result, as shown in FIG. 13, the emission intensity linearly increased as the concentration of the ALP-labeled antibody increased. From this, it was found that when a high concentration ALP-labeled antibody solution is used, non-specific adsorption to the magnetic particle holding carrier increases.

Then, from the emission intensity in the presence of PSA, the emission intensity in the absence of PSA was subtracted to obtain a value (specific signal), which was further divided by the emission intensity (non-specific signal) in the absence of PSA. As a result, when a 10 μg/ml antibody solution was used, the highest value was obtained. A high ratio of specific signal/nonspecific signal means that a high specific signal can be obtained while suppressing a non-specific signal to lower a detection limit. Therefore, when the ALP-labeled antibody is added hereinafter, a 10 μg/ml antibody solution will be used.

(3) Sandwich Immunoassay Using a Magnetic Particle Holding Carrier According to an Embodiment of the Present Invention

As shown in FIG. 11, to the antibody-immobilized magnetic particle holding carrier 63 (2.0×10⁶ beads) having the mouse-derived anti-human PSA antibody 60 immobilized thereto and shown in the step S44, PSA solutions (40 μl) different in concentration were added in the step S45 to prepare beads of the PSA-bound antibody-immobilized magnetic particle holding carrier 65. After the beads of the carrier were washed 3 times with PBST (10 mM PBS, 0.05% tween 20), a solution (10 μg/ml, 40 μl) of the ALP-labeled mouse-derived anti-human PSA antibody 66 was added to prepare the beads of ALP-labeled PSA-bound antibody-immobilized magnetic particle holding carrier 67. After the beads were washed 3 times with PBST, they were suspended in a Tris-HCl buffer solution (5 μl). To this, lumiphos 530 (3.3×10⁻⁴ mol/l, 100 μl) was added to measure emission intensity.

As a result, as shown in FIG. 14, when emission intensity was measured by setting the number of beads (the number of the beads of ALP-labeled PSA-bound antibody-immobilized magnetic particle holding carrier 67) to be suspended in a 100 mM Tris-HCl buffer solution at 1.0×10⁵, 2.0×10⁵ and 4.0×10⁵, a calibration curve exhibited a linear line when 4.0×10⁵ beads were used. It was considered that the calibration range is 0.1 to 10 ng/ml. The blood PSA level of a healthy adult male is less than 3 ng/ml. When a person has a blood PSA level larger than this, the person may have a disease such as prostatic cancer. From this, it was suggested that the sandwich immunoassay using the magnetic particle holding carrier can be applied to diagnosis of prostatic cancer.

As is explained above, based on the observations by a fluorescent microscope and a scanning electron microscope and flow cytometric analysis, it was suggested that biotin/fluorescent dye-introduced bacterial magnetic particles can be efficiently constructed on micro-beads by adding 100 μl of a suspension solution (50 μg/ml) of biotin/fluorescent dye-introduced bacterial magnetic particles (Cy3-BMP-biotin) 10 times to 500 μl of a suspension solution of the streptavidin-labeled micro-size particulate carrier (3.0×10⁶ beads/ml). In addition, it was demonstrated that a 93.9% of beads of the magnetic particle holding carrier can be magnetically separated from the suspension solution by bringing an Nd—B magnet into contact with the upper wall surface of a tube containing the beads of the magnetic particle holding carrier prepared. When an antibody was immobilized onto the biotin/fluorescent dye-introduced bacterial magnetic particles (Cy3-[ZZ-BMP]-biotin) labeled by use of 0.35 mM or 0.035 mM solution mixture of Sulfo-NHS-LC-LC-biotin and Cy3 bis NHS ester, the activity thereof was confirmed. Furthermore, when a magnetic particle holding carrier was prepared by use of antibody-immobilized biotin/fluorescent dye-introduced bacterial magnetic particles (Cy3, biotin-[ZZ-BMP]-Antibody) having an antibody immobilized thereon, it was confirmed that the same structure was constructed as in the case of using biotin/fluorescent dye-introduced bacterial magnetic particles (Cy3-[ZZ-BMP]-Antibody) having no antibody immobilized thereon. It was demonstrated that the antibody immobilized onto the bacterial magnetic particles (BMPs) do not inhibit preparation of the magnetic particle holding carrier.

Then, whether or not the magnetic particle holding carrier according to the embodiment of the present invention is suitable for use in automatization of a treatment will be explained by use of a magnetic particle holding carrier treatment apparatus (a part of a full automatic immunoassay apparatus SX-8PC, manufactured by Precision System Science Co., Ltd. is) shown in FIG. 15 as a treatment-automatization apparatus.

The magnetic particle holding carrier treatment apparatus shown in FIG. 15 has a container group 72 having a plurality of containers 71 (or wells) containing a magnetic particle holding carrier, component substances of the magnetic particle holding carrier such as a carrier or magnetic particles, specimens and requisite reagents; a nozzle head (not shown) having one or two or more nozzles; a tip 75, which has an inlet/outlet 73 for liquid and a fitting cuff 74 to the nozzle and can storing liquid therein; a nozzle (not shown), which fits the fitting cuff 74 of the tip 75 at the distal end thereof and can suction and discharge a gas; a permanent magnet 76 detachably provided to the tip 75, which can apply and remove a magnetic field to the tip 75 externally from the outside the tip 75, or a magnetizable or demagnetizable electromagnet (not shown); a moving means (not shown), which can move the nozzle head in relative to the container group 72; and a control unit (not shown), which controls transfer, separation and resuspension of the magnetic particle holding carrier or its components, a reagent, a specimen and the like by instructing suction or discharge of a suspension solution having the magnetic particle holding carrier or its components suspended in a predetermined solution depending upon the purpose of a treatment, the magnetic particle holding carrier or its components, the reagent and the specimen, or the properties of the suspension solution and which instructs the application or deapplication of the magnetic field in the tip. The suction or discharge instruction by the control unit may include, for example, determination of the flow rate and pressure during suctioning and discharging operations.

### [Experiment 2]

The magnetic particle holding carrier is stable during magnetic separation process carried out in the magnetic particle holding carrier treatment apparatus as described below.

A suspension solution (50 μg/ml, 2 ml) of biotin/fluorescent dye-introduced bacterial magnetic particles 37 (Cy3-BMP-biotin) was added to a suspension solution (3.0×10⁶ beads/ml, 10 ml) of the streptavidin-labeled polystyrene beads, which were micro-size (5 μm in diameter herein) polystyrene beads whose surfaces were coated with streptavidin 13 serving as a receptor and which served as a particulate carrier 32. A reaction was performed for 15 minutes while maintaining a dispersion state by pipetting. This operation was repeated 10 times to prepare the magnetic particle holding carrier 31 according to the third embodiment of the present invention.

Subsequently, using the magnetic particle holding carrier 31, the stability of the biotin/fluorescent dye-introduced bacterial magnetic particles 37 (Cy3-BMP-biotin) on the magnetic particle holding carrier 31 during a magnetic separation process was evaluated.

As shown in FIG. 15, in a step S51, to a predetermined well (container) 71a placed the well (container) group 72 of the magnetic particle holding carrier treatment apparatus and containing a suspension solution of the magnetic particle holding carrier 31, the tip 75 having the permanent magnet 76 detachably provided at the outside thereof is moved by use of the moving means (not shown). In a step S52, the tip 75 is allowed to insert in the well 71a by use of the moving means and the suspension solution is repeatedly suctioned and discharged in the state where the permanent magnet 76 is approached. In this manner, beads of the magnetic particle holding carrier 31 are adsorbed on its inner surface, thereby separating the beads of the magnetic particle holding carrier 31. Subsequently, in a step S53, the tip 75 is transferred to a well 71b, which is arranged next to the well 71a and contains a predetermined solution. In a step S54, the solution is repeatedly suctioned and discharged in the state where the tip 75 is inserted in the well 71b by the moving means In this manner, the beads of magnetic particle holding carrier 31 are resuspended in the well 71b. In a step S55, the tip 75 is removed from the well 71b by use of the moving means. The steps S51 to S55 are repeated 1 to 5 times.

The magnetic particle holding carrier 31 thus obtained and the magnetic particle holding carrier 31 that is not subjected to the aforementioned operation, were subjected to flow cytometry to measure fluorescent intensity. At this time, the number of beads of the magnetic particle holding carrier 31 before magnetic separation was set at 1.5×10⁷ and 150 μl of phosphate buffered saline (PBS) was dispensed to each well.

FIG. 16 shows 7 fluorescent intensity histograms exhibiting the effect upon the magnetic particle holding carrier 31 produced by magnetic separation/resuspension by use of the magnetic particle holding carrier treatment apparatus, in short, stability of the magnetic particle holding carrier 31. In each of the graphs, the horizontal axis represents fluorescent intensity, whereas the vertical axis represents the frequency. FIG. 16(a) shows the fluorescence intensity histogram of polystyrene beads serving as the particulate carrier 32 before holding the biotin/fluorescent dye-introduced bacterial magnetic particles 37 (Cy3-BMP-biotin). FIG. 16(b) shows the fluorescence intensity histogram of the magnetic particle holding carrier 31 of the step S51 before magnetic separation is performed. FIG. 16(c) shows the fluorescence intensity histogram of the magnetic particle holding carrier 31 to which the whole operation from the step S51 to the step S55 is applied once. FIGS. 16(c), (d), (e), (f) and (g) show fluorescence intensity histograms of the magnetic particle holding carrier 31 to which the whole operation is applied 1, 2, 3, 4 and 5 times, respectively. The histogram of the magnetic particle holding carrier 31 shown in FIG. 16(b) shifts toward the right hand side compared to the histogram of the particulate carrier 32 before holding the biotin/fluorescent dye-introduced bacterial magnetic particles 37 shown in FIG. 16(a). From this fact, it was confirmed that the biotin/fluorescent dye-introduced bacterial magnetic particles 37 are held on the particulate carrier 32 (streptavidin-labeled polystyrene). Furthermore, when the fluorescent intensity histogram of the magnetic particle holding carrier 31 before magnetic separation shown in FIG. 16(b) is compared to the fluorescent intensity histograms (FIGS. 16(c) to 16(g)) of the magnetic particle holding carrier 31 to which magnetic separation/resuspension treatment is applied 1 to 5 times, respectively, peaks were observed at the same fluorescent intensity. From the results, it was demonstrated that the amounts of the biotin/fluorescent dye-introduced bacterial magnetic particles 37 (Cy3-BMP-biotin) assembled on the magnetic particle holding carrier 31, even through the magnetic separation was applied 1 to 5 times, are almost the same as that before magnetic separation.

### [Experiment 3]

Next, the magnetic separation ratio of the magnetic particle holding carrier by the magnetic particle holding carrier treatment apparatus (full automatic immunoassay apparatus SX-8PC) is evaluated. The magnetic particle holding carrier 31 was magnetically separated and resuspended repeatedly (1 to 5 times) in the same manner as in Experiment 1 and transferred to the well 71b in the next step. At that time, the concentration of the magnetic particle holding carrier 31 was measured. At this time, the number of the beads of the magnetic particle holding carrier 31 before magnetic separation was set at 1.0×10⁷. As a buffer solution for suspending the magnetic particle holding carrier 31, PBS (2001) containing 0.05% nonionic surfactant, Adekanol (ADK) was used. The magnetic separation rate was obtained based on the following equation:

Magnetic separation rate=concentration of the beads of magnetic particle holding carrier after magnetic separation/concentration of the beads of magnetic particle holding carrier before magnetic separation×100(%).

The magnetic separation rates of 5 magnetic separation operations were obtained. As a result, a recovery rate of 95% or more in average was obtained as shown in FIG. 17. It can be expected that measurement can be accurately performed in a detection operation such as immunoassay.

Next, as a method for preparing the magnetic particle holding carrier 81 according to the fifth embodiment, accumulation by a chemical bonding method using a crosslinking agent 88 having Sulfo-LC-SPDP and Sulfo-SMCC will be explained with reference to FIG. 18.

A magnetic particle holding carrier 81 was prepared by binding bacterial magnetic particles (BMPs) 84 having an amino group 87 displayed thereon to a particulate carrier 82 with the help of a crosslinking agent 88 having Sulfo-LC-SPDP and Sulfo-SMCC, as follows.

The beads of a particulate carrier 82 of 1.0×10⁷ (AP-60-10, having a diameter of 6 to 8 μm, manufactured by Spherotech, Inc.) having an amino group 83 displayed thereon were centrifugally separated at 20400 G for 10 minutes. Thereafter, 0.1M Tris HCl buffer solution (pH 7.0) containing 100 μg of the crosslinking agent component 88b (Sulfo-SMCC) was added. A reaction was performed at room temperature for one hour. The beads were washed 3 times with 1 ml of PBS to obtain the beads of crosslinking agent component introduced particulate carrier 89.

On the other hand, to a carbonate buffer solution (pH 8.5), the crosslinking agent component 88a (Sulfo-LC-SPDP), fluorescent dye (Cy3 bis NHS ester) were dissolved so as to obtain concentrations of 10 mM and 0.1 mM, respectively to obtain a solution mixture. To 500 μl of the solution mixture, 500 μg of bacterial magnetic particles (BMPs) 84 were suspended. A reaction was performed at room temperature for one hour while maintaining a dispersion state by applying ultrasonic treatment at intervals of 5 minutes. Thereafter, the particles were washed 3 times with PBS. To the resultant particles, 2 ml of Tris buffer solution (pH 8.5) containing 20 mM dithiothreitol was added. A reaction was performed at room temperature for 30 minutes. The particles were washed 3 times with 1 ml of PBS to obtain fluorescent dye/crosslinking agent component introduced bacterial magnetic particles 90 (Cy3-BMPs-Sulfo-LC-SPDP).

A suspension solution (50 μg/ml, 100 μl) of the fluorescent dye/crosslinking agent component introduced bacterial magnetic particles 90 (Cy3-BMPs-Sulfo-LC-SPDP) was added to the suspension solution (3.0×10⁶ beads/ml, 500 μl) of the beads of the crosslinking agent component introduced particulate carrier 89 (Sulfo-SMCC-polystyrene beads). A reaction was performed for 15 minutes while maintaining a dispersion state by pipetting. This operation was repeated 1 times to prepare beads of the magnetic particle holding carrier 81. The beads were observed one by one by a fluorescent microscope before the suspension solution of the fluorescent dye/crosslinking agent component introduced bacterial magnetic particles 90 (Cy3-BMPs-Sulfo-LC-SPDP) was added.

As a result, accumulation of the fluorescent dye/crosslinking agent component introduced bacterial magnetic particles 90 (Cy3-BMPs-Sulfo-LC-SPDP) on the beads (polystyrene micro-beads) of the particulate carrier 82 was observed as shown in a microscopic observation images taken in each of the reaction steps of FIG. 19. From this, it was demonstrated that the magnetic particle holding carrier 81 can be formed by a chemical bonding method using a crosslinking agent 88 having Sulfo-LC-SPDP and Sulfo-SMCC.

The reagents and instruments used in the above are as follows:

As the crosslinking agent, sulfosuccinimidyl-6′-(biotinamido)-6-hexanamido hexanote (Sulfo-NHS-LC-LC-Biotin) (purchased from PIERCE); and as the fluorescent dye, Cy3 bis NHS ester (purchased from Amersham Biosciences) were used. As the micro-beads, Streptavidin Polystyrene Particles of 5.0-5.9 μm manufactured by Spherotech, Inc. were used. As the antibody for use in immunoassay, rabbit-derived anti-goat IgG antibody was purchased from SIGMA-ALDRICH and alkaline phosphatase (ALP)-labeled rabbit-derived anti goat IgG antibody from SANTA CRUZ BIOTECHNOLOGY, Inc., and ALP-labeled goat-derived anti-mouse IgG antibody from Beckman Coulter Inc. Furthermore, as a luminescent substrate of ALP, Lumigen PPD, 4-Methoxy-4(3-phosphatephenyl)spiro[1,2-dioxeteane-3,2′adamantane]disodium salt (lumiphos 530: 3.3×10⁻⁴M) manufactured by Wako Pure Chemical Industries was used. When bacterial magnetic transformant cells were cultured and crushed, an ampicillin sodium salt and protease inhibitor cocktail was used. Other reagents all were special-grade commercially available products for research use or quasi-products. Reagents and the like were prepared by use of distilled water or ultrapure water prepared by processing distilled water through MilliQ Lab manufactured by Millipore Corporation.

Collection of magnetic bacterial cells and washing of bacterial magnetic particles (BMPs) were performed by a high-speed centrifugal machine CX-210 manufactured by Tomy Seiko Co., Ltd., a French press 5501M manufactured by Otake Seisakusho Ltd. and an ultrasonic cleaner, SU-25 manufactured by Sibata Scientific Technology Ltd. Bacterial magnetic particles (BMPs) and micro-beads were observed by use of a system biological microscope BX51 manufactured by Olympus Corporation and a scanning electron microscope S-2250N manufactured by Hitachi High-Technologies Corporation. Furthermore, the fluorescent intensity distribution of the magnetic particle holding carrier prepared was analyzed by automatic cell analysis fractionation apparatus (flow cytometer), EPICS ALTRA, manufactured by Beckman Coulter Inc. Emission intensity was measured by use of luminometer Lucy-2 manufactured by Aloka Co., Ltd. The beads of a magnetic particle holding carrier were separated and concentrated by a micro-amount high-speed cooling centrifugal machine, MX-300 and TX-160 manufactured by Tomy Seiko Co., Ltd. Automatic magnetic separation of magnetic particles was performed by a fullautomatic immunoassay apparatus SX-8PC manufactured by Precision System Science Co., Ltd.

In the description above, explanation has been made by taking an amino group or a carboxyl group as a functional group and streptavidin as a receptor, biotin as a ligand, and Cy3 as a fluorescent dye as examples. However, the present invention is not limited to these substances. Other functional groups, various receptors, ligands, fluorescent dyes and marker substances exemplified in the specification may be used. Furthermore, magnetic particles are not limited to bacterial magnetic particles. Magnetic particles to be used may be formed not only by magnetic bacteria but also by coating a magnetic substance with various substances. As a micro-size nonmagnetic particulate carrier, latex and polystyrene carriers have been explained. However, needless to say, other materials exemplified in the specification such as acrylic resin made of acrylic acid, methacrylic acid and derivatives thereof can be used. Furthermore, in the aforementioned examples, the case where a ZZ domain is expressed on bacterial magnetic particles has been explained. However, bacterial magnetic particles can express not only the ZZ domain but also other functional proteins such as Protein A and Protein G. In addition, to this, various types of functional proteins such as an antibody and an antigen can be introduced.

### INDUSTRIAL APPLICABILITY

The present invention relates to a magnetic particle holding carrier and a method for preparing the same. The present invention is concerned with all variety of fields requiring examination or analysis regarding a biological substance, such as the industrial field, agriculture and fisheries field including food, agricultural products and marine products, pharmaceutical field, medical field including hygiene, health, immunity, disease and heredity and science fields including chemistry and biology.

### BRIEF DESCRIPTION OF THE DRAWINGS

FIG. 1 is a schematic illustration showing magnetic particle holding carriers according to embodiments of the present invention;

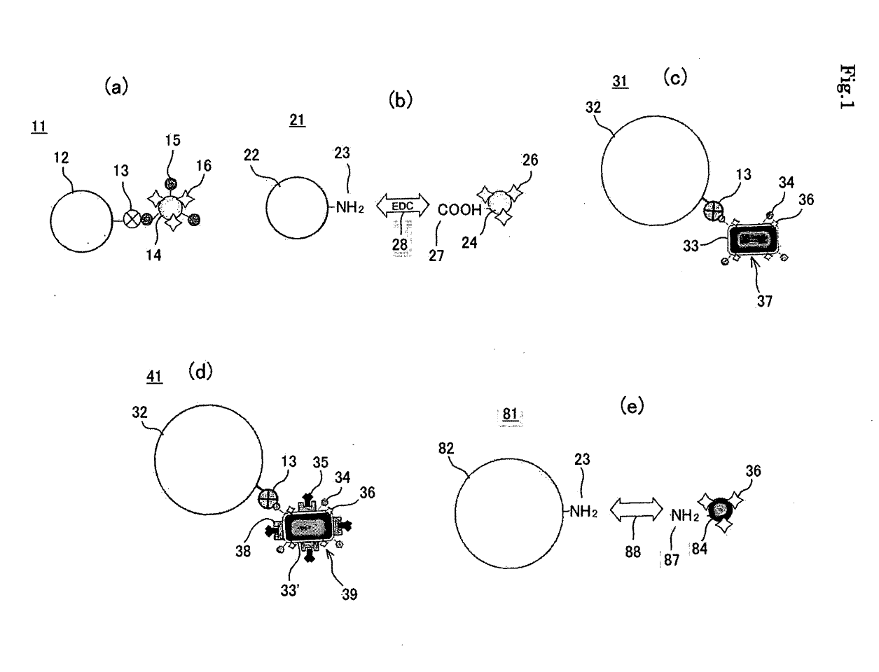

FIG. 2 is an illustration showing the steps of a method for preparing a magnetic particle holding carrier according to an embodiment of the present invention, sequentially in order;

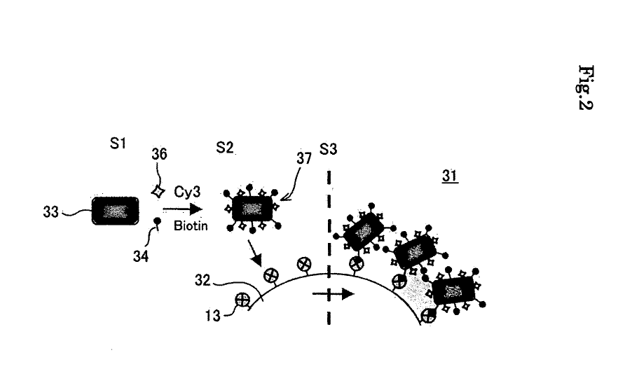

FIG. 3 shows measurement by a fluorescent microscope of a magnetic particle holding carrier according to an embodiment of the present invention, flow cytometric analysis and the measurement results by a scanning electron microscope;

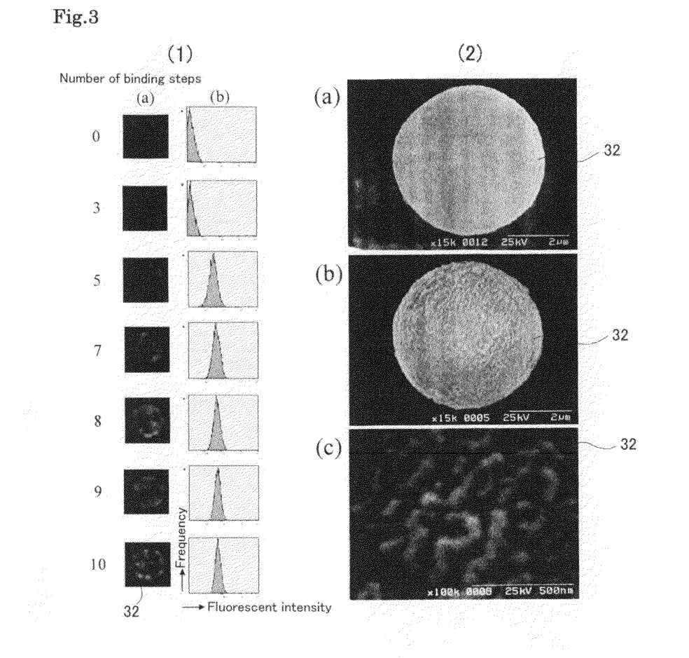

FIG. 4 is an illustration showing a method of determining magnetic separation rate of a magnetic particle holding carrier according to an embodiment of the present invention;

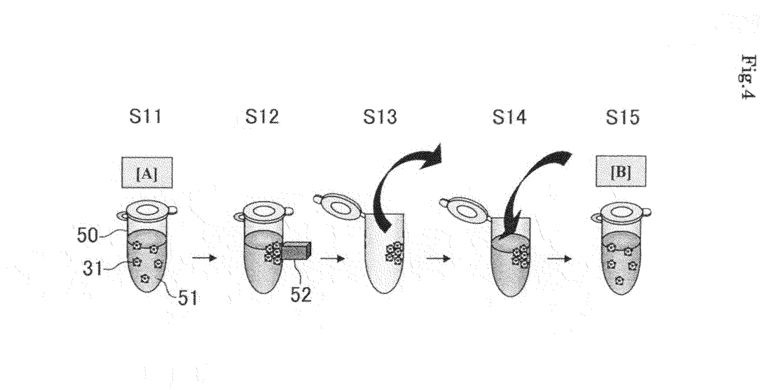

FIG. 5 is a graph showing the measurement results of magnetic separation rate of a magnetic particle holding carrier according to an embodiment of the present invention;

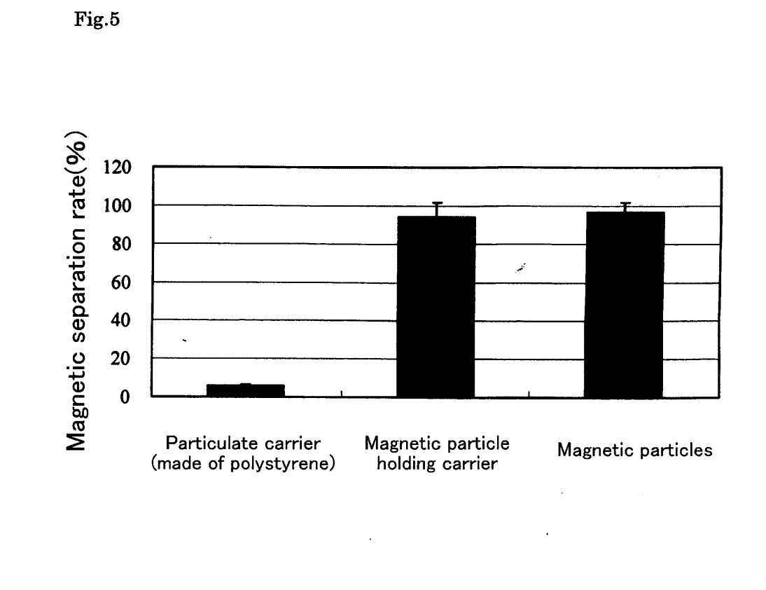

FIG. 6 shows the steps of a method for introducing an antibody into a biotin/fluorescent dye-introduced bacterial magnetic particle according to an embodiment of the present invention, sequentially in order;

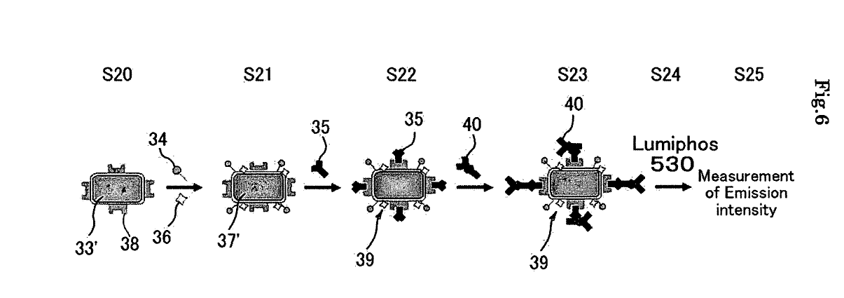

FIG. 7 is a graph showing the amount of antigen bound to antibody-immobilized biotin/fluorescent dye-introduced bacterial magnetic particles according to an embodiment of the present invention;

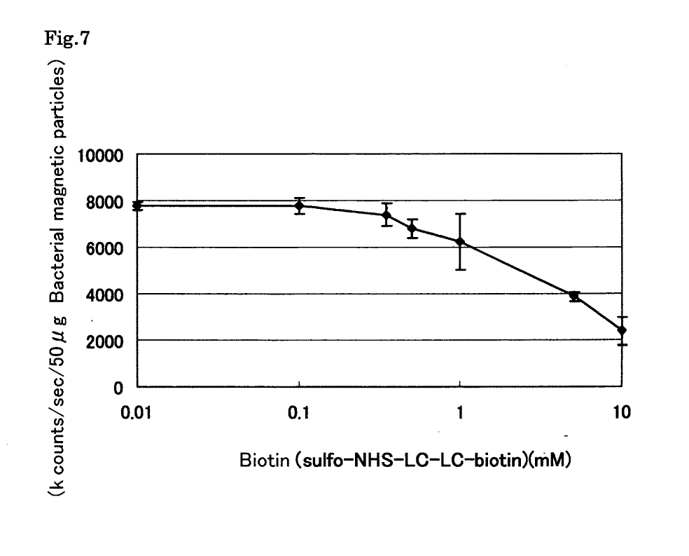

FIG. 8 shows microscopic and flow-cytometric evaluation results of an antibody-immobilized magnetic particle holding carriers according to an embodiment of the present invention;

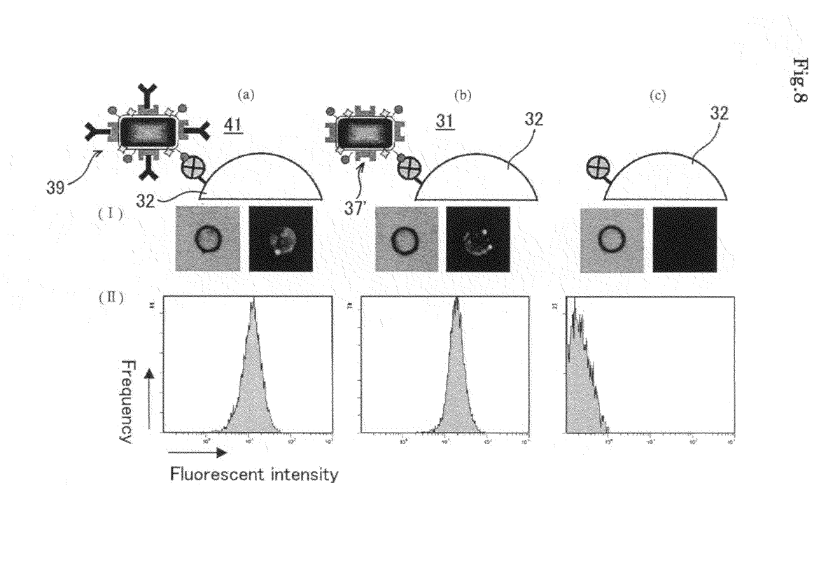

FIG. 9 is an illustration showing the steps of a method for measuring the activity of the antibody of an antibody-immobilized magnetic particle holding carrier according to an embodiment of the present invention, sequentially in order;

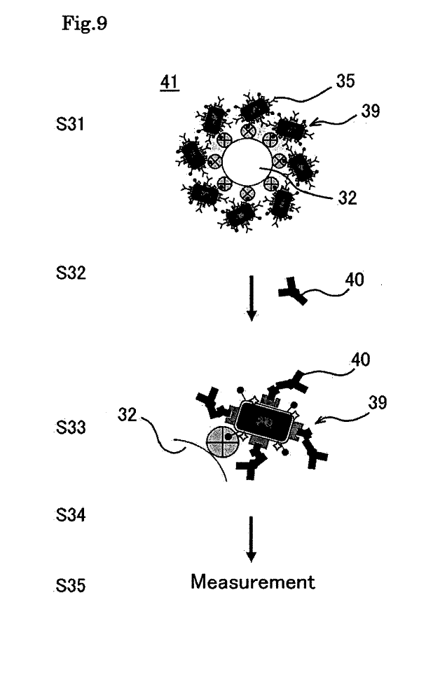

FIG. 10 shows the measurement results of the antibody of an antibody-immobilized magnetic particle holding carrier according to an embodiment of the present invention;

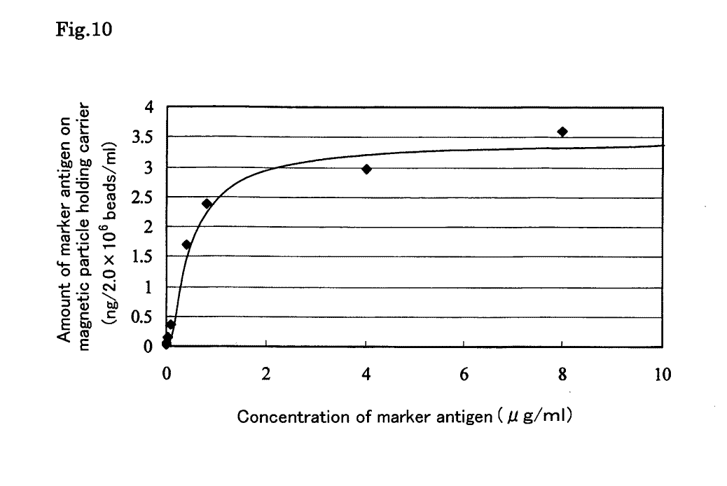

FIG. 11 is an illustration showing the steps of a PSA detection method using a magnetic particle holding carrier according to an embodiment of the present invention, sequentially in order;

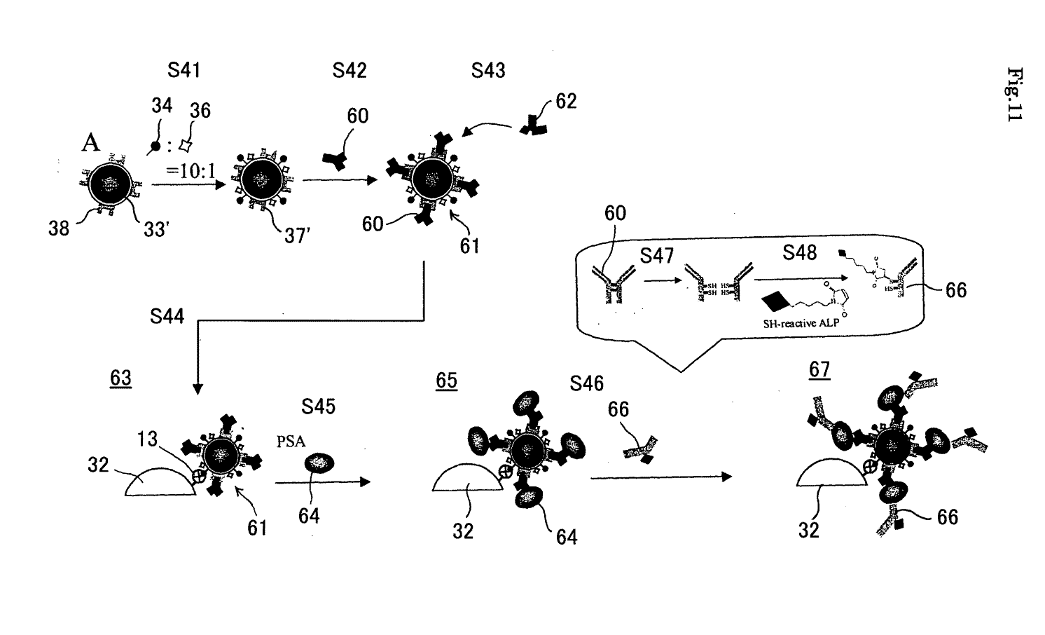

FIG. 12 is a graph showing the relationship between the concentration of immobilized antibody and emission intensity for use in determining the concentration of immobilized antibodies on bacterial magnetic particles according to an embodiment of the present invention;

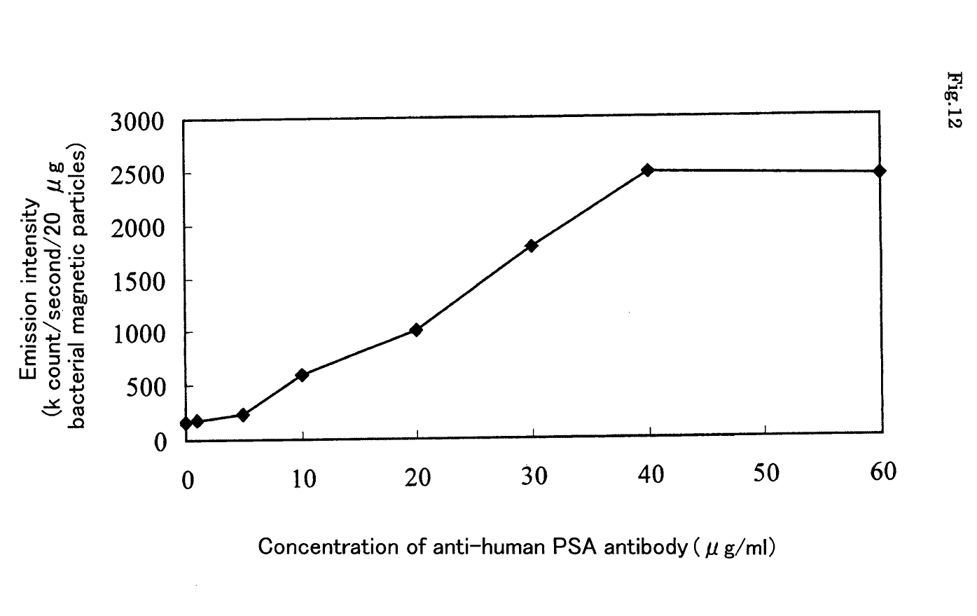

FIG. 13 is a graph showing emission intensity measured in each of the cases where an ALP-labeled antibody solutions different in concentration are added to antibody-immobilized magnetic particle holding carrier according to an embodiment of the present invention;

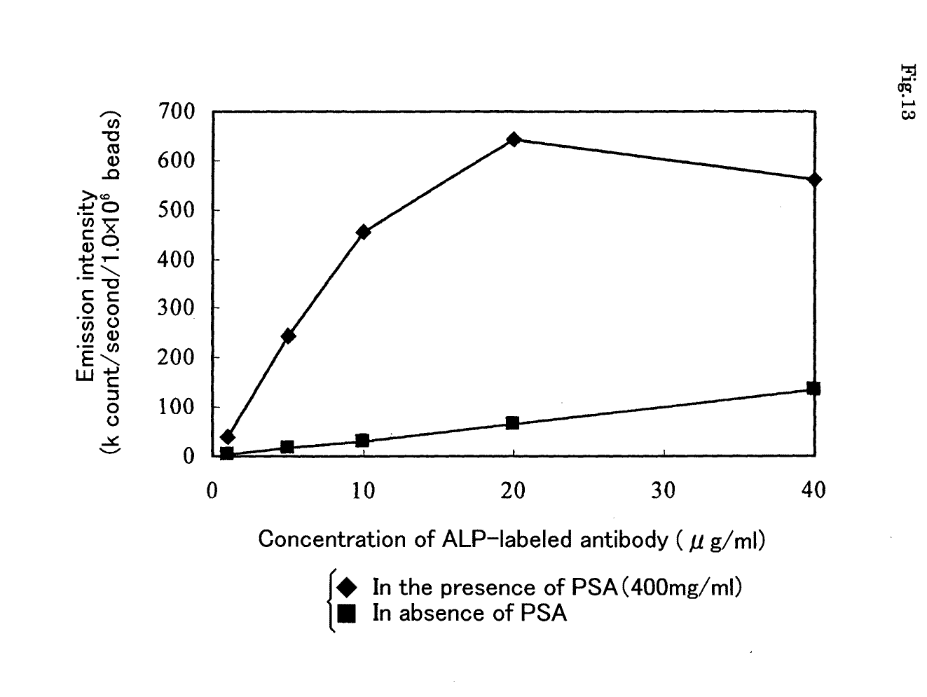

FIG. 14 is a graph showing emission intensity relative to the PSA concentration for detecting PSA by sandwich immunoassay using a magnetic particle holding carrier according to an embodiment of the present invention;

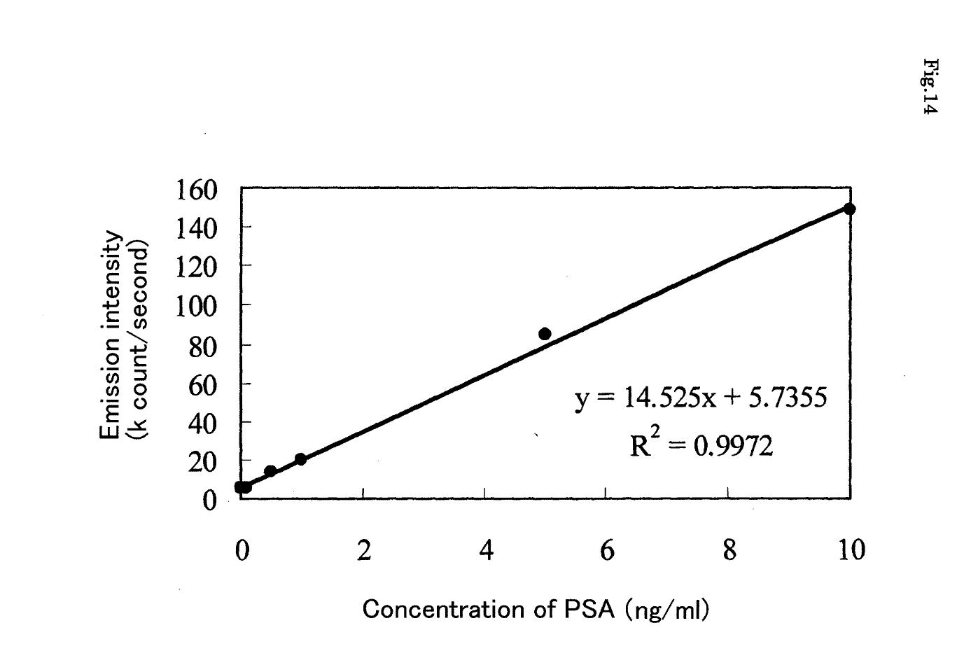

FIG. 15 shows a treatment apparatus for performing a treatment of a magnetic particle holding carrier according to an embodiment of the present invention and shows a magnetic separation/resuspension treatment;

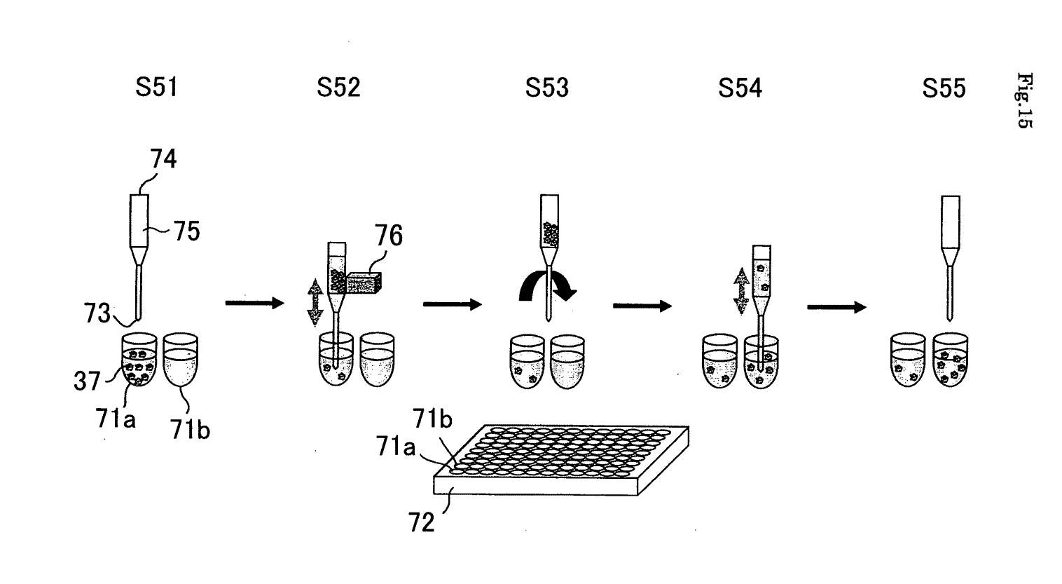

FIG. 16 shows fluorescent intensity histograms showing stability of a magnetic particle holding carrier according to an embodiment of the present invention;

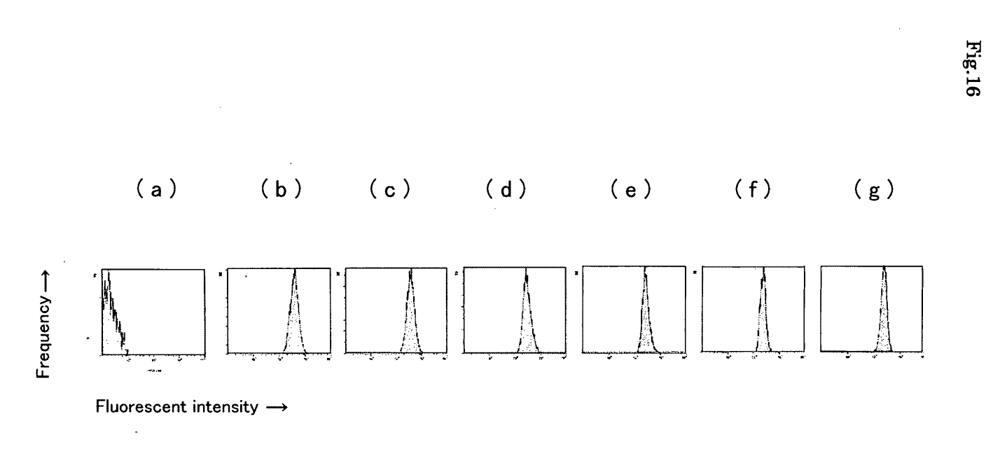

FIG. 17 is a graph showing the magnetic separation efficiency of a magnetic particle holding carrier according to an embodiment of the present invention;

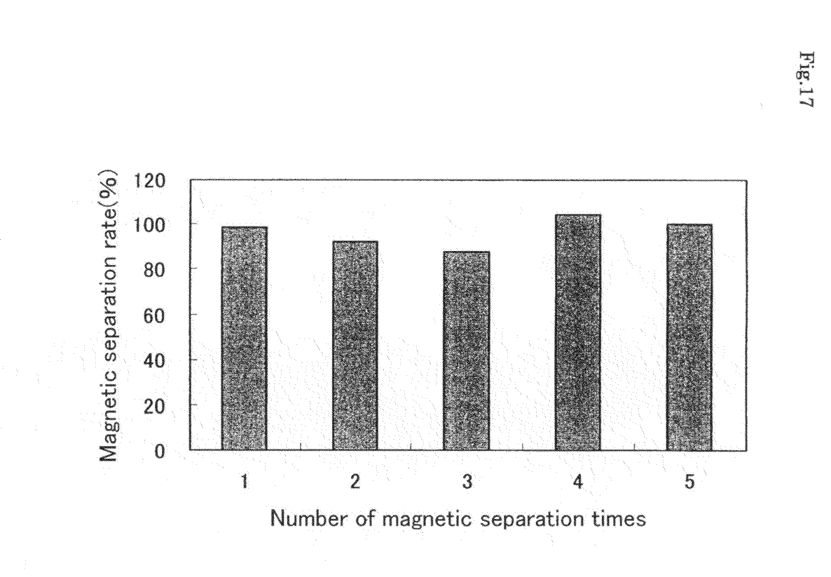

FIG. 18 shows the steps of a method for preparing a magnetic particle holding carrier according to an embodiment of the present invention by use of a chemical bonding method, sequentially in order;

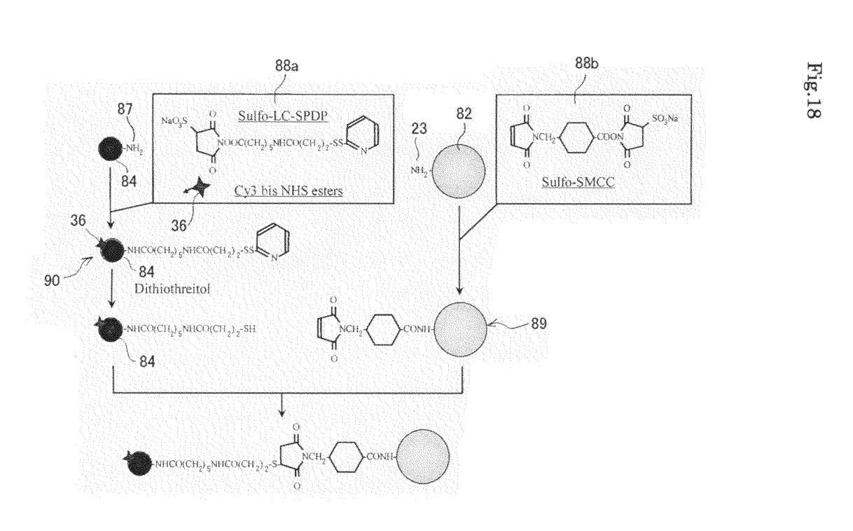

FIG. 19 shows a microscopic image in each reaction step of a method for preparing a magnetic particle holding carrier according to an embodiment of the present invention by use of a chemical bonding method; and

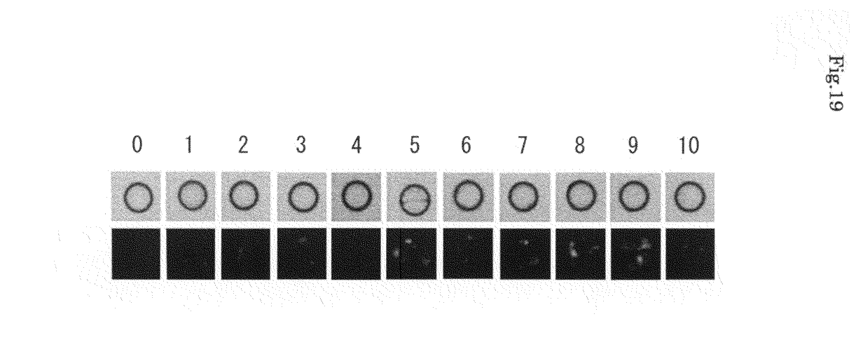

FIG. 20 shows microscopic images of a magnetic particle holding carrier according to an embodiment of the present invention after it is suspended in various dispersion mediums and allowed to stand still for 1 and 48 hours.

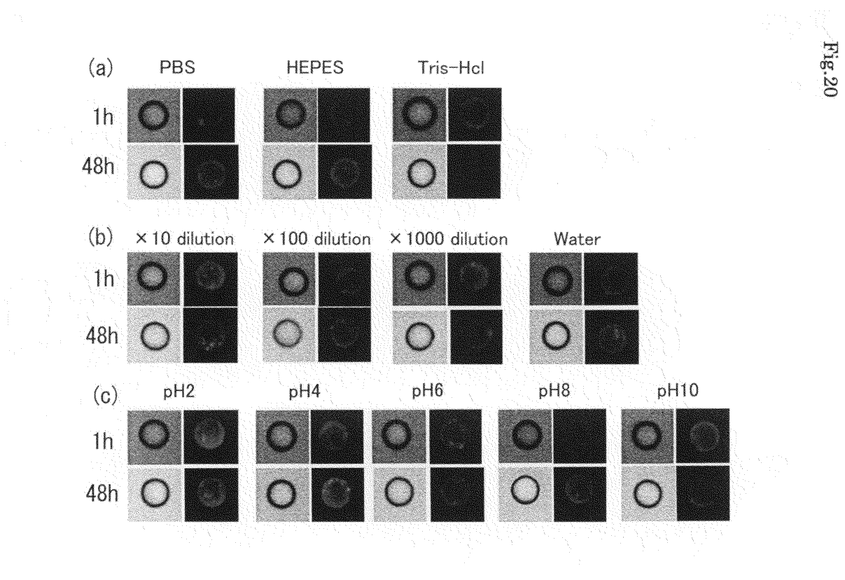

### DESCRIPTION OF SYMBOLS

11, 21, 31, 41, 63, 65, 67, 81 Magnetic particle holding carrier
12, 22, 32, 82 Particulate carrier
14, 24 Super-magnetic single domain particle (magnetic particle)
33, 33′, 84 Bacterial magnetic particle (magnetic particle)
37, 37′ Biotin/fluorescent dye-introduced bacterial magnetic particle
39, 61 Antibody-immobilized biotin/fluorescent dye-introduced bacterial magnetic particle
90 Fluorescent dye/crosslinking agent component introduced bacterial magnetic particle

1. A magnetic particle holding carrier comprising a micro-size nonmagnetic particulate carrier and a plurality of nano-size magnetic particles held on the carrier so as to cover the surface thereof, wherein the magnetic particles are formed of bacterial magnetic particles and the bacterial magnetic particles express a predetermined functional peptide or a functional protein.

2. (canceled)

3. The magnetic particle holding carrier according to claim 1, wherein the carrier has a ligand or a receptor on a surface thereof, the bacterial magnetic particles has a corresponding receptor or ligand, and the bacterial magnetic particles are held on the carrier by ligand-receptor binding.

4. The magnetic particle holding carrier according to claim 1, wherein the bacterial magnetic particles are held on the carrier via a covalent bond, hydrogen bond or electrostatic bond.

5. The magnetic particle holding carrier according to claim 1, wherein the bacterial magnetic particles have a single type or a plurality of types of marker substances.

6. The magnetic particle holding carrier according to claim 5, wherein the marker substance(s) has a ligand or a receptor and the bacterial magnetic particles have a corresponding receptor or ligand, the marker substance(s) is introduced into the bacterial magnetic particles by ligand-receptor binding.

7. The magnetic particle holding carrier according to claim 5, wherein the marker substance(s) is introduced into the bacterial magnetic particles via a covalent bond, hydrogen bond or electrostatic bond.

8. The magnetic particle holding carrier according to claim 5, wherein the receptor or the ligand is expressed on the bacterial magnetic particles.

9. The magnetic particle holding carrier according to claim 1, wherein the bacterial magnetic particles are isolated from a magnetic bacterium.

10. A method for preparing a magnetic particle holding carrier having a plurality of nano-size magnetic particles on a micro-size nonmagnetic particulate carrier so as to cover the surface thereof, comprising:
a process step for applying processing to the magnetic particles and/or the carrier; and
a suspension step for suspending the magnetic particles and a plurality of carriers in liquids
wherein the magnetic particles are formed of bacterial magnetic particles and the process step has an expression step for permitting the bacterial magnetic particles to express a predetermined functional peptide or functional protein.

11. (canceled)

12. The method for preparing a magnetic particle holding carrier according to claim 10, wherein the process step has an introduction-into-carrier step for introducing a ligand or a receptor into the carrier and/or an introduction-into-magnetic-particle step for introducing a corresponding receptor or ligand into the bacterial magnetic particles.

13. The method for preparing a magnetic particle holding carrier according to claim 10, wherein, in the suspension step, covalent bonding, hydrogen bonding or electrostatic bonding is performed.

14. The method for preparing a magnetic particle holding carrier according to claim 10, wherein the process step has a step of introducing a marker substance into the bacterial magnetic particles.

15. The method for preparing a magnetic particle holding carrier according to claim 14, comprising a step of introducing a ligand or a receptor into the marker substance and/or a step of introducing a corresponding receptor or ligand into the bacterial magnetic particles.

16. The method for preparing a magnetic particle holding carrier according to claim 15, wherein, in the process step, covalent bonding, hydrogen bonding or electrostatic bonding is performed.

17. The method for preparing a magnetic particle holding carrier according to claim 15, wherein the process step has a step of expressing the receptor or ligand on the bacterial magnetic particles.

18. The method for preparing a magnetic particle holding carrier according to claim 10, further comprising an isolation step for isolating the bacterial magnetic particles from a magnetic bacterium.

19. The magnetic particle holding carrier according to claim 1, wherein the functional peptide or the functional protein is fused with an anchor protein expressed on a lipid bilayer covering an outer surface of the bacterial magnetic particles.
